SEQUENCE LISTING (93 sequences)

SEQ ID NO: 1 - DNA, 36 bp, Homo sapiens
tggctagccgccaccatgaccatgaccctccacacc

SEQ ID NO: 2 - DNA, 29 bp, Homo sapiens
gactcgagtcagaccgtggcagggaaacc

SEQ ID NO: 3 - DNA, 36 bp, Homo sapiens
tggctagccgccaccatggttttggaaatgctgaac

SEQ ID NO: 4 - DNA, 29 bp, Homo sapiens
cctctagactagtgttccagacacctgtc

SEQ ID NO: 5 - DNA, 39 bp, Homo sapiens
gcgttcgctaagcgtagctagcgatgtacgggccagata

SEQ ID NO: 6 - DNA, 21 bp, Homo sapiens
gcgttcgctaagcgtagctag

SEQ ID NO: 7 - DNA, 42 bp, Homo sapiens
tctgatacgtctcgacgcactctcccagcatgcctgctattg

SEQ ID NO: 8 - DNA, 22 bp, Homo sapiens
tctgatacgtctcgacgcactc

SEQ ID NO: 9 - DNA, 39 bp, Homo sapiens
atgatcaaacgctctaagaggaacagcctggccttgtcc

SEQ ID NO: 10 - DNA, 39 bp, Homo sapiens; identical to SEQ ID NO: 9

SEQ ID NO: 11 - DNA, 39 bp, Homo sapiens; identical to SEQ ID NO: 9

SEQ ID NO: 12 - DNA, 39 bp, Homo sapiens
catctccagcagcaggtcagagaggggcaccacgttctt

SEQ ID NO: 13 - DNA, 39 bp, Homo sapiens
aaacgctctaagaagaacgagctggccttgtccctgacg

SEQ ID NO: 14 - DNA, 39 bp, Homo sapiens
cgtcagggacaaggccagctcgttcttcttagagcgttt

SEQ ID NO: 15 - DNA, 39 bp, Homo sapiens
ttactgaccaacctggcatacagggagctggttcacatg

SEQ ID NO: 16 - DNA, 39 bp, Homo sapiens; identical to SEQ ID NO: 15

SEQ ID NO: 17 - DNA, 39 bp, Homo sapiens
gaagcttcgatgatgggcgctttactgaccaacctggca

SEQ ID NO: 18 - DNA, 39 bp, Homo sapiens
tgccaggttggtcagtaaagcgcccatcatcgaagcttc

SEQ ID NO: 19 - DNA, 39 bp, Homo sapiens
gatcaggtccaccttctacaatgtgcctggctagagatc

SEQ ID NO: 20 - DNA, 39 bp, Homo sapiens
gatctctagccaggcacattgtagaaggtggacctgatc

SEQ ID NO: 21 - DNA, 39 bp, Homo sapiens
ggagtgtacacatttctgcccagcaccctgaagtctctg

SEQ ID NO: 22 - DNA, 39 bp, Homo sapiens
cagagacttcagggtgctgggcagaaatgtgtacactcc

SEQ ID NO: 23 - DNA, 39 bp, Homo sapiens
ctgcagcagcagcaccagtggctggcccagctcctcctc

SEQ ID NO: 24 - DNA, 39 bp, Homo sapiens
gaggaggagctgggccagccactggtgctgctgctgcag

SEQ ID NO: 25 - DNA, 39 bp, Homo sapiens
atgaagtgcaagaacgtggagcccctctatgacctgctg

SEQ ID NO: 26 - DNA, 39 bp, Homo sapiens
cagcaggtcatagaggggctccacgttcttgcacttcat

SEQ ID NO: 27 - DNA, 39 bp, Homo sapiens
aagtgcaagaacgtggtgcacctctatgacctgctgctg

SEQ ID NO: 28 - DNA, 39 bp, Homo sapiens
cagcagcaggtcatagaggtgcaccacgttcttgcactt

SEQ ID NO: 29 - DNA, 39 bp, Homo sapiens
tgcaagaacgtggtgcccccctatgacctgctgctggag

SEQ ID NO: 30 - DNA, 39 bp, Homo sapiens
ctccagcagcaggtcataggggggcaccacgttcttgca

SEQ ID NO: 31 - DNA, 39 bp, Homo sapiens
tgcaagaacgtggtgccccgctatgacctgctgctggag

SEQ ID NO: 32 - DNA, 39 bp, Homo sapiens
ctccagcagcaggtcatagcggggcaccacgttcttgca

SEQ ID NO: 33 - DNA, 39 bp, Homo sapiens
tgcaagaacgtggtgccccagtatgacctgctgctggag

SEQ ID NO: 34 - DNA, 39 bp, Homo sapiens
ctccagcagcaggtcatactggggcaccacgttcttgca

SEQ ID NO: 35 - DNA, 39 bp, Homo sapiens
aacgtggtgcccctctatggcctgctgctggagatgctg

SEQ ID NO: 36 - DNA, 39 bp, Homo sapiens
cagcatctccagcagcaggccatagaggggcaccacgtt

SEQ ID NO: 37 - DNA, 39 bp, Homo sapiens
aagaacgtggtgcccctctgtgacctgctgctggagatg

SEQ ID NO: 38 - DNA, 39 bp, Homo sapiens
catctccagcagcaggtcacagaggggcaccacgttctt

SEQ ID NO: 39 - DNA, 39 bp, Homo sapiens
aagaacgtggtgcccctcaatgacctgctgctggagatg

SEQ ID NO: 40 - DNA, 38 bp, Homo sapiens
catctccagcagcaggtcattgaggggcaccacgttct

SEQ ID NO: 41 - DNA, 39 bp, Homo sapiens
cggctccgcaaatgctacaaagtgggaatgatgaaaggt

SEQ ID NO: 42 - DNA, 39 bp, Homo sapiens
acctttcatcattcccactttgtagcatttgcggagccg

SEQ ID NO: 43 - DNA, 39 bp, Homo sapiens
ccaaattcagataatcgatgccagggtggcagagaaaga

SEQ ID NO: 44 - DNA, 39 bp, Homo sapiens
tctttctctgccaccctggcatcgattatctgaatttgg

SEQ ID NO: 45 - DNA, 39 bp, Homo sapiens
tgccaggcctgccggctctgcaaatgctacgaagtggga

SEQ ID NO: 46 - DNA, 39 bp, Homo sapiens
tcccacttcgtagcatttgcagagccggcaggcctggca

SEQ ID NO: 47 - DNA, 39 bp, Homo sapiens
ggggcatccgtggaggagatggaccaaagccacttggcc

SEQ ID NO: 48 - DNA, 39 bp, Homo sapiens
ggccaagtggctttggtccatctcctccacggatgcccc

SEQ ID NO: 49 - DNA, 39 bp, Homo sapiens
agcatgaagtgcaagaacatggtgcccctctatgacctg

SEQ ID NO: 50 - DNA, 39 bp, Homo sapiens
caggtcatagaggggcaccatgttcttgcacttcatgct

SEQ ID NO: 51 - DNA, 39 bp, Homo sapiens
tgtgtagagggcatggtggatatcttcgacatgctgctg

SEQ ID NO: 52 - DNA, 39 bp, Homo sapiens
cagcagcatgtcgaagatatccaccatgccctctacaca

SEQ ID NO: 53 - DNA, 39 bp, Homo sapiens
agaatgttgaaacacaagtgccagagagatgatggggag

SEQ ID NO: 54 - DNA, 39 bp, Homo sapiens
ctccccatcatctctctggcacttgtgtttcaacattct

SEQ ID NO: 55 - DNA, 39 bp, Homo sapiens
ggtttccccccactcaacaacgtgtctccgagcccgctg

SEQ ID NO: 56 - DNA, 39 bp, Homo sapiens
cagcgggctcggagacacgttgttgagtggggggaaacc

SEQ ID NO: 57 - DNA, 39 bp, Homo sapiens
ctacatgcgcccactagccatggaggggcatccgtggag

SEQ ID NO: 58 - DNA, 39 bp, Homo sapiens
ctccacggatgcccctccatggctagtgggcgcatgtag

SEQ ID NO: 59 - DNA, 39 bp, Homo sapiens
ctgctggctacatcatcttggttccgcatgatgaatctg

SEQ ID NO: 60 - DNA, 39 bp, Homo sapiens
cagattcatcatgcggaaccaagatgatgtagccagcag

SEQ ID NO: 61 - DNA, 39 bp, Homo sapiens
aaccagggaaaatgtgtagatggcatggtggagatcttc

SEQ ID NO: 62 - DNA, 38 bp, Homo sapiens
gaagatctccaccatgccatctacacattttccctggt

SEQ ID NO: 63 - DNA, 39 bp, Homo sapiens
atggaatctgccaaggaggctcgctactgtgcagtgtgc

SEQ ID NO: 64 - DNA, 39 bp, Homo sapiens
gcacactgcacagtagcgagcctccttggcagattccat

SEQ ID NO: 65 - DNA, 39 bp, Homo sapiens
atgaagtgcaagaacgtgatgcccctctatgacctgctg

SEQ ID NO: 66 - DNA, 39 bp, Homo sapiens
cagcaggtcatagaggggcatcacgttcttgcacttcat

SEQ ID NO: 67 - DNA, 39 bp, Homo sapiens; identical to SEQ ID NO: 31

SEQ ID NO: 68 - DNA, 39 bp, Homo sapiens; identical to SEQ ID NO: 32

SEQ ID NO: 69 - DNA, 39 bp, Homo sapiens
accatgaccatgaccctctacaccaaagcatctgggatg

SEQ ID NO: 70 - DNA, 39 bp, Homo sapiens
catcccagatgctttggtgtagagggtcatggtcatggt

SEQ ID NO: 71 - DNA, 39 bp, Homo sapiens
ggtggcagagaaagattgtccagtaccaatgacaaggga

SEQ ID NO: 72 - DNA, 39 bp, Homo sapiens
tcccttgtcattggtactggacaatctttctctgccacc

SEQ ID NO: 73 - DNA, 39 bp, Homo sapiens
gagatgctggacgcccaccacctacatgcgcccactagc

SEQ ID NO: 74 - DNA, 39 bp, Homo sapiens
gctagtgggcgcatgtaggtggtgggcgtccagcatctc

SEQ ID NO: 75 - DNA, 39 bp, Homo sapiens
atcctgatgattggtctcatctggcgctccatggagcac

SEQ ID NO: 76 - DNA, 39 bp, Homo sapiens; identical to SEQ ID NO: 75

SEQ ID NO: 77 - DNA, 39 bp, Homo sapiens
gacgcccaccgcctacatgtgcccactagccgtggaggg

SEQ ID NO: 78 - DNA, 39 bp, Homo sapiens
ccctccacggctagtgggcacatgtaggcggtgggcgtc

SEQ ID NO: 79 - DNA, 39 bp, Homo sapiens; identical to SEQ ID NO: 5

SEQ ID NO: 80 - DNA, 21 bp, Homo sapiens
gcgttcgctaagcgtagctag

SEQ ID NO: 81 - DNA, 42 bp, Homo sapiens; identical to SEQ ID NO: 7

SEQ ID NO: 82 - DNA, 22 bp, Homo sapiens
tctgatacgtctcgacgcactc

SEQ ID NO: 83 - DNA, 39 bp, Homo sapiens
gacgtgttgacccttctgcatcgtgtcatgctggacacc

SEQ ID NO: 84 - DNA, 39 bp, Homo sapiens
ggtgtccagcatgacacgatgcagaagggtcaacacgtc

SEQ ID NO: 85 - DNA, 39 bp, Homo sapiens
ccatttggctttgggccctgtggctgtgcaggaaagtac

SEQ ID NO: 86 - DNA, 39 bp, Homo sapiens
gtactttcctgcacagccacagggcccaaagccaaatgg

SEQ ID NO: 87 - DNA, 69 bp, Homo sapiens
tcgaggtcaggtcacagtgacctgatgtcaggtcacagtgacctgatgtcaggtcacagtgacctgata

SEQ ID NO: 88 - DNA, 69 bp, Homo sapiens
agcttatcaggtcactgtgacctgacatcaggtcactgtgacctgacatcaggtcactgtgacctgacc

SEQ ID NO: 89 - DNA, 1788 bp, Homo sapiens
atgaccatgaccctccacaccaaagcatctgggatggccctactgcatcagatccaagggaacgagctggagcccctgaaccgtccgcagctcaagatccccctggagcggcccctgggcgaggtgtacctggacagcagcaagcccgccgtgtacaactaccccgagggcgccgcctacgagttcaacgccgcggccgccgccaacgcgcaggtctacggtcagaccggcctcccctacggccccgggtctgaggctgcggcgttcggctccaacggcctggggggtttccccccactcaacagcgtgtctccgagcccgctgatgctactgcacccgccgccgcagctgtcgcctttcctgcagccccacggccagcaggtgccctactacctggagaacgagcccagcggctacacggtgcgcgaggccggcccgccggcattctacaggccaaattcagataatcgacgccagggtggcagagaaagattggccagtaccaatgacaagggaagtatggctatggaatctgccaaggagactcgctactgtgcagtgtgcaatgactatgcttcaggctaccattatggagtctggtcctgtgagggctgcaaggccttcttcaagagaagtattcaaggacataacgactatatgtgtccagccaccaaccagtgcaccattgataaaaacaggaggaagagctgccaggcctgccggctccgcaaatgctacgaagtgggaatgatgaaaggtgggatacgaaaagaccgaagaggagggagaatgttgaaacacaagcgccagagagatgatggggagggcaggggtgaagtggggtctgctggagacatgagagctgccaacctttggccaagcccgctcatgatcaaacgctctaagaagaacagcctggccttgtccctgacggccgaccagatggtcagtgccttgttggatgctgagccccccatactctattccgagtatgatcctaccagacccttcagtgaagcttcgatgatgggcttactgaccaacctggcagacagggagctggttcacatgatcaactgggcgaagagggtgccaggctttgtggatttgaccctccatgatcaggtccaccttctagaatgtgcctggctagagatcctgatgattggtctcgtctggcgctccatggagcacccagggaagctactgtttgctcctaacttgctcttggacaggaaccagggaaaatgtgtagagggcatggtggagatcttcgacatgctgctggctacatcatctcggttccgcatgatgaatctgcagggagaggagtttgtgtgcctcaaatctattattttgcttaattctggagtgtacacatttctgtccagcaccctgaagtctctggaagagaaggaccatatccaccgagtcctggacaagatcacagacactttgatccacctgatggccaaggcaggcctgaccctgcagcagcagcaccagcggctggcccagctcctcctcatcctctcccacatcaggcacatgagtaacaaaggcatggagcatctgtacagcatgaagtgcaagaacgtggtgcccctctatgacctgctgctggagatgctggacgcccaccgcctacatgcgcccactagccgtggaggggcatccgtggaggagacggaccaaagccacttggccactgcgggctctacttcatcgcattccttgcaaaagtattacatcacgggggaggcagagggtttccctgccacggtctga

SEQ ID NO: 90 - protein, 595 aa, Homo sapiens [sequence omitted]

SEQ ID NO: 91 - DNA, 1502 bp, Homo sapiens
atggttttggaaatgctgaacccgatacattataacatcaccagcatcgtgcctgaagccatgcctgctgccaccatgccagtcctgctcctcactggcctttttctcttggtgtggaattatgagggcacatcctcaataccaggtcctggctactgcatgggaattggacccctcatctcccacggcagattcctgtggatggggatcggcagtgcctgcaactactacaaccgggtatatggagaattcatgcgagtctggatctctggagaggaaacactcattatcagcaagtcctcaagtatgttccacataatgaagcacaatcattacagctctcgattcggcagcaaacttgggctgcagtgcatcggtatgcatgagaaaggcatcatatttaacaacaatccagagctctggaaaacaactcgacccttctttatgaaagctctgtcaggccccggccttgttcgtatggtcacagtctgtgctgaatccctcaaaacacatctggacaggttggaggaggtgaccaatgaatcgggctatgtggacgtgttgacccttctgcgtcgtgtcatgctggacacctctaacacgctcttcttgaggatccctttggacgaaagtgctatcgtggttaaaatccaaggttattttgatgcatggcaagctctcctcatcaaaccagacatcttctttaagatttcttggctatacaaaaagtatgagaagtctgtcaaggatttgaaagatgccatagaagttctgatagcagaaaaaagacgcaggatttccacagaagagaaactggagactttgccactgagttgattttagcagagaaacgtggtgacctgacaagagagaatgtgaaccagtgcatattggaaatgctgatcgcagctcctgacaccatgtctgtctctttgttcttcatgctatttctcattgcaaagcaccctaatgttgaagaggcaataataaaggaaatccagactgttattggtgagagagacataaagattgatgatatacaaaaattaaaagtgatggaaaacttcatttatgagagcatgcggtaccagcctgtcgtggacttggtcatgcgcaaagccttagaagatgatgtaatcgatggctacccagtgaaaaaggggacaaacattatcctgaatattggaaggatgcacagactcgagtttttccccaaacccaatgaatttactcttgaaaattttgcaaagaatgttccttataggtactttcagccatttggctttgggccccgtggctgtgcaggaaagtacatcgccatggtgatgatgaaagccatcctcgttacacttctgagacgattccacgtgaagacattgcaaggacagtgtgttgagagcatacagaagatacacgacttgtccttgcacccagatgagactaaaaacatgctggaaatgatctttaccccaagaaactcagacaggtgtctggaacactag

SEQ ID NO: 92 - protein, 503 aa, Homo sapiens
MVLEMLNPIHYNITSIVPEAMPAATMPVLLLTGLFLLVWNYEGTSSIPGPGYCMGIGPLISHGRFLWMGIGSACNYYNRVYGEFMRVWISGEETLIISKSSSMFHIMKHNHYSSRFGSKLGLQCIGMHEKGIIFNNNPELWKTTRPFFMKALSGPGLVRMVTVCAESLKTHLDRLEEVTNESGYVDVLTLLRRVMLDTSNTLFLRIPLDESAIVVKIQGYFDAWQALLIKPDIFFKISWLYKKYEKSVKDLKDAIEVLIAEKRRRISTEEKLEECMDFATELILAEKRGDLTRENVNQCILEMLIAAPDTMSVSLFFMLFLIAKHPNVEEAIIKEIQTVIGERDIKIDDIQKLKVMENFIYESMRYQPVVDLVMRKALEDDVIDGYPVKKGTNIILNIGRMHRLEFFPKPNEFTLENFAKNVPYRYFQPFGFGPRGCAGKYIAMVMMKAILVTLLRRFHVKTLQGQCVESIQKIHDLSLHPDETKNMLEMIFTPRNSDRCLEH

SEQ ID NO: 93 - DNA, 21 bp, Homo sapiens
gtcaggtcacagtgacctgat

(19) United States
(12) Patent Application Publication
(10) Pub. No.: US 2018/0217131 A1
(43) Pub. Date: Aug. 2, 2018

(54) Novel Cell Based Assay

(71) Applicant: Medical Diagnostic Laboratories, LLC, Hamilton, NJ (US)

(72) Inventor: Yick Loi Raymond Yu, East Brunswick, NJ (US)

(73) Assignee: Medical Diagnostic Laboratories, LLC, Hamilton, NJ (US)

(21) Appl. No.: 15/418,006

(22) Filed: Jan. 27, 2017

(51) Int. Cl. G01N 33/50; C12Q 1/68

(52) U.S. Cl. CPC G01N 33/5041; G01N 2333/90245; G01N 2333/723; C12Q 1/6897

(57) ABSTRACT

The present invention relates to cell-based assays involving the estrogen receptor and/or aromatase. The assays use assay cells that are transfected with linear cassettes containing the ESR1 or the CYP19A1 gene of interest and measure the effect of mutations on the activity of the estrogen receptor and/or aromatase, and on their response to inhibitors.

Filed even date herewith via the EFS-Web is an ASCII text file containing the sequence listing, which is named “MDL_00065_sequence_listing_text”, was created on Jan. 20, 2017, and contains 27 kilobytes; said ASCII text file is incorporated herein by reference.

### FIELD OF THE INVENTION

The present invention generally relates to cell-based assays involving the estrogen receptor and/or aromatase. These cell-based assays are used to measure the effect of mutations on the activity of the estrogen receptor and/or aromatase, and on their responses to inhibitors.

### BACKGROUND OF THE INVENTION

Breast cancer is the most prevalent cancer in women, and over two-thirds of cases express estrogen receptor a (encoded by ESR1). The estrogen receptor a, also known as NR3A1, (referred to herein as “ER”) is a member of the nuclear receptor family and regulates the transformed phenotype of the majority of breast cancers. ER is the primary therapeutic target in breast cancer. Drugs directly antagonizing ER, including selective estrogen receptor modulators and selective estrogen receptor degraders, are a mainstay of breast cancer treatment. In addition to anti-estrogen therapies, patient with ER-positive breast cancer are also treated with aromatase inhibitors (referred to herein as “AI”s). Aromatase (referred to herein as “AR”) is the enzyme that catalyzes the conversion of androgens, in particular testosterone, into estrogen in vivo. AR is encoded by CYP19A1. AIs block the peripheral conversion of androgens into estrogen and, in post-menopausal women, lead to over a 98% reduction in circulating levels of estrogen. Although most patients with ER-positive breast cancer derive a benefit from these drugs, resistance often emerges after prolonged exposure.

Cancer evolution and progression are driven by a sequence of somatic genetic and non-genetic alterations resulting in more favorable tumor cell growth and survival. Cancer genetic evolution is subject to intrinsic influences such as the tumor microenvironment, as well as extrinsic pressures such as drug therapy. The clinical pattern of acquired resistance may, in many circumstances, represent outgrowth of resistant clones, which may have originally been present in the cancer at low frequency as a result of intra-tumoral genetic heterogeneity, but grow out under the selective pressure of targeted therapy.

Advances in high-throughput sequencing technologies are beginning to establish a molecular taxonomy for a spectrum of human disease and has facilitated a move toward precision medicine. With regard to oncology, defining the mutational landscape of a patient's tumor will lead to more precise treatment and management of individuals with cancer. In addition to the potential for identifying ‘actionable’ therapeutic targets in cancer patients, the clinical sequencing may also shed light on acquired resistance mechanisms developed against targeted therapies. Although uncovering the DNA sequence of tumor becomes possible with the advances in next generation of sequencing, this technology does not provide any functional information of the identified mutations. Therefore, there is a need to develop a novel method to study the functional consequence of mutation on the target gene activity and its response toward the drug treatment. This functional information will provide additional valuable guidelines for the physician to choose the most appropriate treatment based on the mutational landscape of patient's tumor.

Several methods related to ER or AR activity have been published. One such method involves a MCF-7 cell line expressing ER wild type and overexpressing AR wild type which is transfected via a plasmid with a luciferase reporter gene construct to screen for AR inhibitors and ERα ligands (see, Lui et al., “MCF-7aro/ERE, a Novel Cell Line for Rapid Screening of Aromatase Inhibitors, ERα Ligands and ERRα Ligands”, Biochem Pharmacol., 2008 Jul. 15; 76(2): 208-215); however, there is no disclosure of assessing gene mutations. Another publication describes a transient luciferase reporter assay to assess the function of certain gene mutants (see, Robinson, et al., “Activating ESR1 mutations in hormone-resistant metastatic breast cancer”, Nat Genet. 2013 December; 45(12): 1446-1451); however, the assay cells were not stably transfected with the reporter and the mutant ESR1 genes were transfected via the lentiviral vector pCDH.

Accordingly, there is a continuing need to develop an improved clinical test using a cell-based assay that measures ER or AR activity useful in assessing the effect of somatic ESR1 and CYP19A1 gene mutations in the respective protein activity, as well as determining the sensitivity of ER and AR variants to inhibitors.

### SUMMARY OF THE INVENTION

In one aspect the present invention concerns a method of determining whether an ER variant is sensitive to treatment with an ER inhibitor in a cell, comprising the steps of:
a) preparing a cDNA encoding said ER variant;
b) preparing a linear expression cassette containing the ER variant cDNA;
c) transfecting said prepared linear expression cassette containing said ER variant cDNA in an assay cell having a stably integrated signal expression construct comprising a reporter gene cDNA linked to an ERE sequence;
d) exposing said transfected cell to an estrogen, wherein said estrogen complexes with said ER variant which thereby binds to said ERE sequence on the signal construct and generates a signal;
e) exposing said transfected cell with an ER inhibitor; and
f) determining whether said ER variant is sensitive to treatment with said ER inhibitor by measuring a change in signal.

In another aspect the present invention concerns A method of determining the activity of an ER variant, comprising the steps of:
a) preparing a cDNA encoding said ER variant;
b) preparing a linear expression cassette containing the ER variant cDNA;
c) transfecting said prepared linear expression cassette containing said ER variant cDNA in a cell having a stably integrated signal expression construct comprising a reporter gene cDNA linked to an ERE sequence;
d) exposing said transfected cell to an estrogen, wherein said estrogen complexes with said ER variant which thereby binds to said ERE sequence on the signal construct and generates a signal;
e) determining the signal activity, wherein a change in signal activity relative to wild type estrogen receptor is indicative of a change in activity of said ER variant.

In another aspect the present invention concerns a method of determining whether the activity of an AR variant is sensitive to treatment with an AR inhibitor in a cell, comprising the steps of:
a) preparing a cDNA encoding said AR variant;
b) preparing a linear expression cassette containing the AR variant cDNA;
c) transfecting said prepared linear expression cassette containing said AR variant cDNA in an assay cell having a stably integrated signal expression construct comprising a reporter gene cDNA linked to an ERE sequence and said assay cell further having a stably integrated expression construct of ER;
d) exposing said transfected cell to an androgen which is converted to estrogen by said AR variant, wherein said estrogen complexes with stably expressed ER which thereby binds to said ERE sequence on the signal construct and generates a signal;
e) exposing said transfected cell with an AR inhibitor; and
f) determining whether said AR variant is sensitive to treatment with said AR inhibitor by measuring a change in signal.

In another aspect the present invention concerns a method of determining the activity of an aromatase variant, comprising the steps of:
a) preparing a cDNA encoding said aromatase variant;
b) preparing a linear expression cassette containing the aromatase variant cDNA;
c) transfecting said prepared linear expression cassette containing said aromatase variant cDNA in an assay cell having a stably integrated signal expression construct comprising a reporter gene cDNA linked to an estrogen response element sequence and said assay cell further having a stably integrated expression construct of estrogen receptor;
d) exposing said transfected cell to an androgen which is converted to estrogen by said aromatase variant, wherein said estrogen complexes with stably expressed estrogen receptor which thereby binds to said estrogen response element sequence on the signal construct and generates a signal;
e) determining the signal activity, wherein a change in signal activity relative to wild type aromatase is indicative of an increase or decrease in activity of said aromatase variant.

### BRIEF DESCRIPTION OF THE DRAWING

FIG. 1 depicts the PCR mediated overlapping extension to construct patient ESR1 gene. Two rounds of PCR were performed. The first round of PCR included two independent PCR as illustrated as PCR#1 and PCR#2. ESR1 expression plasmid was used as DNA template. Second round of PCR was performed after ExoSAP-IT® treatment. Mixture of the PCR products from first round of PCR was used as DNA template. The mutation was incorporated into the final linear expression cassette, which contained CMV promoter and BGH terminator, as illustrated in the figure. “UF” and “UR” refer to sequences for universal forward and universal reverse, respectively.

FIG. 2 depicts the Sanger sequencing result of the linear expression cassette of A) K303R ESR1 mutant and B) G400V/M643A/L544A ESR1 mutant.

FIG. 3 depicts the luciferase reporter activity of HEK 293 cells transfected with active luciferase reporter using reverse and forward transfection methods.

FIG. 4 depicts the immunoblot result using antibody against estrogen receptor. HEK 293 cells were transfected with nothing or linear expression cassette of WT, K303R or G400V/M643A/L544AESR1.

FIG. 5 depicts the luciferase reporter activity of HEK 293 cells transiently transfected with 3×ERE-Luc and pcDNA or 3×ERE-Luc and ESR1 expression plasmid. The cells were also treated with estrogen.

FIG. 6 depicts the luciferase reporter activity of HEK 293 cells transiently transfected with 3×ERE-Luc and ESR1 expression plasmid. The cells were treated with estrogen and estrogen receptor inhibitors.

FIG. 7 depicts the luciferase reporter activity of HEK 293 cells transiently transfected with 3×ERE-Luc and wild-type ESR1 or Y537S ESR1 expression plasmid.

FIG. 8 depicts the luciferase reporter activity of single cell clones stably integrated with 3×ERE-Luc. The cells were transiently transfected with wild-type ESR1 expression plasmid followed by treatment of estrogen.

FIG. 9 depicts the dose response curve of single cell clone #4, #12 and #20. The cells were transiently transfected with wild-type ESR1 expression plasmid followed by treatment of a serial dilution of estrogen.

FIG. 10 depicts the dose response curve of estrogen and fulvestrant for clone #4. The cells were transiently transfected with wild-type ESR1 expression plasmid followed by treatment of a serial dilution of estrogen or treatment of 64 pM of estrogen in the presence of a serial dilution of fulvestrant.

FIG. 11 depicts the dose response curve estrogen for single cell clone #4. The cells were transiently transfected with linear expression cassette of ESR1 followed by treatment of a serial dilution of estrogen.

FIG. 12 depicts the luciferase reporter activity of single cell clone #4. The cells were transiently transfected with 10 ng, 25 ng, 50 ng or 100 ng of linear expression cassette of ESR1 followed by treatment of estrogen.

FIG. 13 depicts the luciferase reporter activity of single cell clone #4. The cells were transiently transfected with 50 ng of linear expression cassette of ESR1 followed by treatment of estrogen for 2 hours, 4 hours and 6 hours.

FIG. 14 depicts the luciferase reporter activity of single cell clone #4 using two different exposure methods. A) Schematic representation of two different exposure methods, 6 hours and “all-in-one”. B) The cells were transfected with 50 ng of linear expression cassette of ESR1 followed by treatment of estrogen for 6 hours. Alternatively, estrogen was added to the cells using the “all-in-one” method.

FIG. 15 depicts the luciferase reporter activity of single cell clone #4 using the cells cultured for either A) 3 days or B) 4 days. The cells were transiently transfected with 50 ng of linear expression cassette of ESR1 followed by treatment of estrogen using the “all-in-one” method.

FIG. 16 depicts the agonistic activity of tamoxifen in single cell clone #4 when the cells were transiently transfected with 50 ng of linear expression cassette of ESR1 following by treatment of different concentration of tamoxifen using the “all-in-one” method.

FIG. 17 depicts the inhibitory activity of tamoxifen using single cell clone #4 transiently transfected with 50 ng of linear expression cassette of ESR1. A) The transfected cells were treated with different concentration of tamoxifen in the presence or absence of 50 pM of estrogen. The luciferase reporter activity was measured and illustrated. B) The percentage activity of estrogen receptor was calculated and summarized in the table.

FIG. 18 depicts the agonistic activity of toremifene in single cell clone #4 when the cells were transiently transfected with 50 ng of linear expression cassette of ESR1 following by treatment of different concentration of toremifene using the “all-in-one” method.

FIG. 19 depicts the inhibitory activity of toremifene using single cell clone #4 transiently transfected with 50 ng of linear expression cassette of ESR1. A) The transfected cells were treated with different concentration of toremifene in the presence or absence of 50 pM of estrogen. The luciferase reporter activity was measured and illustrated. B) The percentage activity of estrogen receptor was calculated and summarized in the table.

FIG. 20 depicts the agonistic activity of raloxifene in single cell clone #4 when the cells were transiently transfected with 50 ng of linear expression cassette of ESR1 following by treatment of different concentration of raloxifene using the “all-in-one” method.

FIG. 21 depicts the inhibitory activity of raloxifene using single cell clone #4 transiently transfected with 50 ng of linear expression cassette of ESR1. A) The transfected cells were treated with different concentration of raloxifene in the presence or absence of 50 pM of estrogen. The luciferase reporter activity was measured and illustrated. B) The percentage activity of estrogen receptor was calculated and summarized in the table.

FIG. 22 depicts the luciferase activity of single cell clone #4 performed on three different types of 384-well plate. The cells were transiently transfected with 50 ng of linear expression cassette of ESR1 followed by treatment of estrogen at different concentration using “all-in-one” method.

FIG. 23 depicts the luciferase activity of single cell clone #4 performed on two different lots of 384-well plate from Corning 3570. The cells were transiently transfected with 50 ng of linear expression cassette of ESR1 followed by treatment of estrogen at different concentration using “all-in-one” method.

FIG. 24 depicts the receptor activity of wild-type ESR1 and the inhibitory activities of tamoxifen, toremifene, raloxifene and fulvestrant using single cell clone #4 transiently transfected with 50 ng of linear expression cassette of wild-type ESR1. The transfected cells were treated with different concentrations of estrogen and estrogen receptor inhibitors using “all-in-method”. A) The luciferase reporter activity was measured and illustrated. B) The percentage activity of estrogen receptor was calculated and summarized in the table.

FIG. 25 depicts the luciferase reporter activity of cells transfected with either 50 ng of linear expression cassette of wild-type ESR1 or 344InsC ESR1 mutant. A) The transfected cells were treated with different concentrations of estrogen. B) The transfected cells were treated with two different concentrations of raloxifene in the presence of 80 pM of estrogen. The percentage of activity of estrogen receptor was illustrated.

FIG. 26 depicts the luciferase reporter activity of cells transfected with either 50 ng of linear expression cassette of wild-type ESR1 or V534E ESR1 mutant. A) The transfected cells were treated with different concentrations of estrogen. B) The transfected cells were treated with estrogen receptor inhibitors in the presence of 80 pM of estrogen. The percentage of activity of estrogen receptor was illustrated.

FIG. 27 depicts the luciferase reporter activity of cells transfected with either 50 ng of linear expression cassette of wild-type ESR1 or E380Q ESR1 mutant. A) The transfected cells were treated with different concentrations of estrogen. B) The transfected cells were treated with estrogen receptor inhibitors in the presence of 80 pM of estrogen. The percentage of activity of estrogen receptor was illustrated.

FIG. 28 depicts the luciferase reporter activity of cells transfected with either 50 ng of linear expression cassette of wild-type ESR1 or L536P ESR1 mutant. A) The transfected cells were treated with different concentrations of estrogen. B) The transfected cells were treated with estrogen receptor inhibitors in the presence of 80 pM of estrogen. The percentage of activity of estrogen receptor was illustrated.

FIG. 29 depicts the Sanger sequencing result of the linear expression cassette of A) Y361F CYP19A1 mutant and B) M374A CYP19A1 mutant.

FIG. 30 depicts the luciferase reporter activity of 3×ERE-luc clone #4 cells, which are stably integrated with 3×ERE-Luc. The cells were transiently transfected with either wild-type ESR1 expression plasmid or expression plasmids of ESR1 and CYP19A1 followed by treatment of testosterone.

FIG. 31 depicts the luciferase reporter activity of 3×ERE-luc clone #4 cells, which are stably integrated with 3×ERE-Luc. The cells were transiently transfected with expression plasmids of ESR1 and CYP19A1 followed by treatment of testosterone and its inhibitors.

FIG. 32 depicts the luciferase reporter activity of 3×ERE-luc clone #4 cells, which are stably integrated with 3×ERE-Luc. The cells were transiently transfected with expression plasmids of wild-type ESR1 and wild-type or mutants of CYP19A1 followed by treatment of testosterone.

FIG. 33 depicts the luciferase reporter activity of single cell clones stably integrated with 3×ERE-Luc and expression plasmid of wild-type ESR1. The single cell clones were treated with testosterone.

FIG. 34 depicts the luciferase reporter activity of clone #1 cells, which are stably integrated with 3×ERE-Luc and expression plasmid of wild-type ESR1. The cells were transiently transfected with 0 ng, 25 ng and 50 ng of linear expression cassette of wild-type CYP19A1 followed by treatment of testosterone.

FIG. 35 A) Schematic representation of exposure methods. B) The luciferase reporter activity of clone #1 cells, which are stably integrated with 3×ERE-Luc and expression plasmid of wild-type ESR1. The cells were transiently transfected with 50 ng of linear expression cassette of wild-type CYP19A1 followed by treatment of testosterone for 4 hours, 6 hours and 24 hours.

FIG. 36 depicts the luciferase reporter activity of clone #1 cells, which are stably integrated with 3×ERE-Luc and expression plasmid of wild-type ESR1. A) Schematic representation of “all-in-one” method. B) The cells were transiently transfected with 50 ng of linear expression cassette of wild-type CYP19A1 followed by treatment of testosterone using “all-in-one” method.

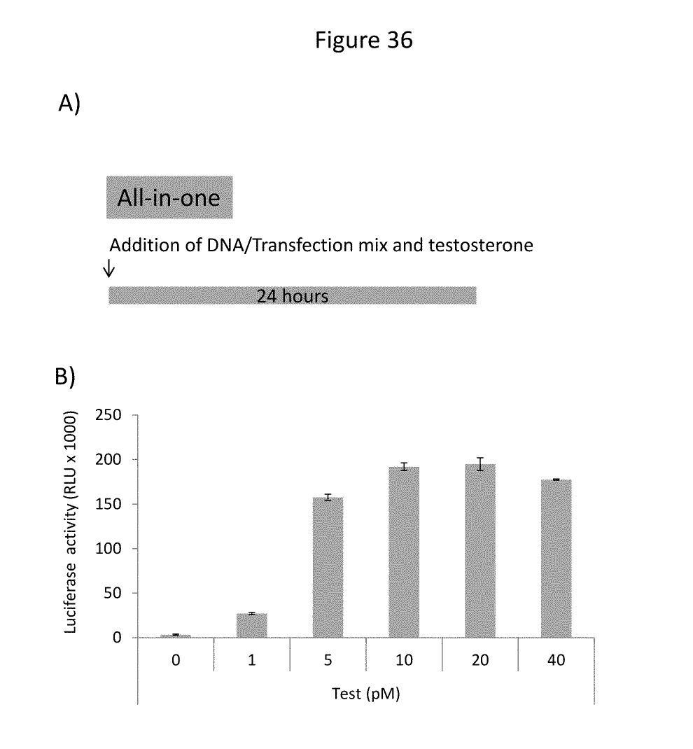

FIG. 37 depicts A) luciferase reporter activity of clone #1 cells, which are stably integrated with 3×ERE-Luc and expression plasmid of wild-type ESR1. The cells were transiently transfected with 50 ng of linear expression cassette of wild-type CYP19A1 followed by treatment of testosterone and a serial dilution of anastrozole using “all-in-one” method. B) The percentage of activity of aromatase was illustrated.

FIG. 38 depicts A) luciferase reporter activity of clone #1 cells, which are stably integrated with 3×ERE-Luc and expression plasmid of wild-type ESR1. The cells were transiently transfected with 50 ng of linear expression cassette of wild-type CYP19A1 followed by treatment of testosterone and a serial dilution of letrozole using “all-in-one” method. B) The percentage of activity of aromatase was illustrated.

FIG. 39 depicts A) luciferase reporter activity of clone #1 cells, which are stably integrated with 3×ERE-Luc and expression plasmid of wild-type ESR1. The cells were transiently transfected with 50 ng of linear expression cassette of wild-type CYP19A1 followed by treatment of testosterone and its inhibitors using “all-in-one” method. B) The percentage of activity of aromatase was illustrated.

FIG. 40 depicts A) luciferase reporter activity of clone #1 cells, which are stably integrated with 3×ERE-Luc and expression plasmid of wild-type ESR1. The cells were transiently transfected with 50 ng of linear expression cassette of R192H mutant of CYP19A1 followed by treatment of testosterone and its inhibitors using “all-in-one” method. B) The percentage of activity of aromatase was illustrated.

FIG. 41 depicts luciferase reporter activity of clone #1 cells, which are stably integrated with 3×ERE-Luc and expression plasmid of wild-type ESR1. The cells were transiently transfected with 50 ng of linear expression cassette of R435C mutant of CYP19A1 followed by treatment of testosterone.

### DETAILED DESCRIPTION OF THE INVENTION

The present invention can be better understood from the following description of preferred embodiments, taken in conjunction with the accompanying drawings. It should be apparent to those skilled in the art that the described embodiments of the present invention provided herein are merely exemplary and illustrative and not limiting. Numerous embodiments or modifications thereof are contemplated as falling within the scope of the present invention and equivalents thereto. All publications, patent applications, patents, and other references mentioned herein are incorporated by reference in their entirety.

In the context of the invention, the term “treating” or “treatment”, as used herein, means reversing, alleviating, mitigating or inhibiting the progress of the disorder or condition to which such term applies, or one or more symptoms of such disorder or condition. As used herein and in the appended claims, the singular forms “a”, “an”, and “the” include plural reference unless the context clearly dictates otherwise.

### Definitions

Various terms used in this specification shall have the definitions set out herein. All derivatives, inflections and conjugations or other grammatical forms of a specific term are intended to be included in the recited definition.

As used herein, the term “A,” “T,” “C”, and “G” refer to adenine, thymine, cytosine, and guanine as a nucleotide base, respectively.

As used herein, the term “ESR1” refers to the gene which transcribes RNA that translates into the ER protein which is exemplified by SEQ ID NO: 89.

As used herein, the term “CYP19A1” refers to the gene which transcribes RNA that translates into the AR protein which is exemplified by SEQ ID NO: 91.

As used herein, the term “ER” refers to the human estrogen receptor

As used herein, the term “AR” refers to human aromatase.

As used herein the term “wild type” or “WT” means the typical form of an organism, strain, gene or characteristic as it occurs in nature as distinguished from mutant, variant, or modified forms.

As used herein the term “variant” means the exhibition of qualities that have a pattern that deviates from what occurs in nature or is distinct from the predominant form that occurs in nature.

As used herein, the term “vehicle” refers to the solvent of a compound.

As used herein, the term “CRISPR” refers to Clustered regularly interspaced short palindromic repeats, which are sequences used by CRISPR associated proteins (Cas) for the purpose of recognizing and cutting genetic elements. CRISPR/Cas9 uses sgRNA as a recognition sequence for identifying where the Cas9 will bind and cut the genetic element.

As used herein, the term “cancer” refers to a malignant neoplastic disease. Most cancers are characterized by hyperproliferation of a cell population.

As referred to herein, the terms “test cell” and “assay cell” may interchangeably be used. The terms are directed to an assay cell which is transfected for use in the assays of the invention.

As used herein, the term “tumor cell” or “cancer cell” refers to a malignant neoplastic cell.

As used herein, the term “luciferase activity” refers to the use of a luciferase protein or reporter to assess the amount of luciferase light emission. The activity is measured by addition of a substrate that binds to the luciferase protein and emits a light signal that can be measured using a luminometer.

As used herein, the term “promoter” refers to a region of the DNA that facilitates the transcription of a particular gene.

As used herein, “expression” or “expressed” refers to the processes by which a polynucleotide is transcribed from a DNA template (such as into and mRNA or other RNA transcript) and further processed or translated into peptides, polypeptides, or proteins. Transcripts and encoded polypeptides may be collectively referred to as “gene product.” If the polynucleotide is derived from genomic DNA, expression may include differential splicing of the mRNA in a eukaryotic cell leading to different forms of peptides or protein products.

As used herein, the terms “stable expression” or “stably expressing” refer to the a cell line or group of cells that express a given protein for a period greater than 1 week, normally resulting in permanent expression of that protein over months.

As used herein, the term “stable cell” or “stable cell system” refers to the generation of cells using a selection method that specifically stably express a given protein. “Stable cell clone” is derived from a single cell with stable expression of a given protein.

As used herein, the term “transfection” refers to the process of introducing a polynucleotide into a cell, and more specifically into the interior of a membrane-enclosed space of a target cell(s), such as the cytosol of a cell, the nucleus of a cell, an interior space of a mitochondria, endoplasmic reticulum (ER), and the like. Transfection can be accomplished by any means known to those of skill in the art, for example as taught by Sambrook et al. Molecular Cloning: A Laboratory Manual, Cold Spring Harbor Laboratory Press, New York (2001), the contents of which are incorporated by reference herein. Examples of transfection techniques include, but are not limited to: heat shock, calcium phosphate transfection, PEI transfection, electroporation, lipofection, transfection reagent(s), viral-mediated transfer, and the like, or combinations thereof.

As used herein, the term “transiently transfected” refers to a cell that has been subject to a process of introducing a polynucleotide into a cell resulting in expression of a protein over a period from 12 hours to 7 days.

As used herein, the term “ER inhibitor” refers to a compound which targets and binds to ER, preventing or reducing ER activity.

As used herein, the term “ERE” refers to the estrogen response element.

As used herein, the term “AR inhibitor” refers to a compound which targets and binds to AR, preventing or reducing AR activity.

As used herein the term “reporter” means a protein that when expressed in a cell is capable of producing a detectable signal.

As used herein the term “reporter gene” refers to a polynucleotide that encodes a reporter.

As used herein the term “signal expression construct” refers to a polynucleotide that contains cDNA encoding a reporter protein, typically an enzyme such as luciferase, and an estrogen response element.

The present invention relates to ER and AR variants sensitivity to treatment, tumor cell sensitivity to treatment, ER and AR variants sensitivity to inhibitors and whether compounds inhibit ER or AR activity, respectively, in a cell. In one aspect, the present invention provides highly sensitive methods for determining whether a ER or AR variant is sensitive to treatment using a ER or AR inhibitor, respectively.

Methods include providing a stable cell system containing cDNA constructs with signaling capability to measure the interaction between ER or AR variants and treatment with inhibitors. The ER or AR variants may include one or more mutations in the respective gene. Identification of the ER and AR variants may be obtained from sequencing of a biological sample or produced using Next Generation Sequencing (NGS).

The assay cells of the invention are stably transfected with a reporter gene linked to ERE. When ER is activated by estrogen, ER migrates or translocates to the nucleus and binds to ERE. When the ER binds to ERE in the assay cells the reporter gene is expressed and a signal is produced. In one embodiment the binding of ER to ERE results in expression of the reporter protein and produces a signal. In some embodiments in order to produce a signal a substrate must be provided for the reporter. The signal can be, for example, a light signal. In some embodiments the reporter is an enzyme which can be any protein produced from any gene that exhibits enzymatic activity and degrades a substrate to produce a light or luminescence signal. The light signal can be measured using a luminometer. In certain embodiments, the light is measured by fluorescence signaling systems such as Fluorescence Resonance Energy Transfer (FRET).

Examples of reporter enzymes include luciferase, alkaline phosphatase, chloramphenicol transferase, β-galactosidase, β-glucuronidase, carboxylesterase, lipases, phospholipases, sulphatases, ureases peptidases, proteases and the like. In a particular embodiment the reporter is luciferase, for example, firefly luciferase, Renilla luciferase, and the like. In one aspect, the present invention provides a method to determine ESR1 or CYP19A1 gene activity using a cell based reporter assay. Ligand-dependent nuclear transactivation involves direct binding of ER to the ERE sequence in the nucleus. In the assays of the invention the stable assay cells contain a reporter construct which comprises a reporter gene linked to ERE. ER binding to ERE results in a signal. In one embodiment ER binding to ERE induces expression of the reporter which leads to generation of light emission when a substrate is added, and therefore allows for indirect measurement of ER or AR activity. Typical substrates for luciferase include D-luciferin and salts thereof.

The present assay can be used in personalized medicine. When genome information is obtained relating to ER or AR sequences, one skilled in the art can conveniently prepare a cDNA based on the ESR1 or CYP19A1gene sequence information. The generated cDNA therefore contains a unique cDNA for that individual because it contains a gene sequence of the specific gene of that individual. In certain embodiments, the cDNA encoding the ER or AR variant may be transiently transfected into the cell. One advantage of the present assay is to transfect the generated ER or AR cDNA into an assay cell with a linear cassette. Compressing or shortening the time required to perform the assays of the invention can be important when performing the assays in a clinical laboratory By use of linear cassette transfection the time required to obtain and report results to a physician can be substantially shortened relative to prior art methods using plasmids. For example performing the assays of the invention (i.e., steps (a) through (f) of the inhibitor sensitivity assays, and steps (a) through (e) of the activity assays) can take 32 hours or less, typically 30 hours or less, preferably 28 hours or less.

Normally, transfection with the linear cassette takes about 4 to 24 hours, in some embodiments about 5 to 20 hours, in another embodiment about 6 to 12 hours. In one embodiment, once the assay cells are transfected with the linear cassette, the assay cells are cultured or incubated under conditions suitable for adequate cell growth and expression of the proteins of interest; in one embodiment the conditions include a culture time of 24 to 48 hours typically at about 37° C. After transfection, the assay cells are exposed to estrogen or androgen for about 2 to 6 hours. If an estrogen inhibitor or AI is used in the assay, the assay cells are exposed to said inhibitor for about 2 to 6 hours. The hormone and inhibitor of the hormone in one embodiment are added to the assay cells at about the same time, i.e., simultaneously. In another embodiment, it has been surprisingly discovered that estrogen or androgen can be added at about the same time as transfection with the linear cassette, i.e., performing steps c) and d) simultaneously (termed herein as the “all-in-one” method), and still result in a good dose response to the respective hormone.

Transfection with the linear expression cassette can be a “forward transfection” or a “reverse transfection.” Forward transfection is where cells are seeded a day prior to transfection in order to achieve an actively dividing cell population adhered to a vessel surface at the time of transfection. Reverse transfection is where freshly passaged cells are added to transfection complexes. Reverse transfection has the advantage of reducing the time for performing assays; however, because the cells are not adhered to the surface of the assay vessel (e.g., surface of the wells of the assay plate) and are not in a robust growth phase, reverse transfection can be unsuitable for certain assays. It has been discovered that for the assays of the invention reverse transfection results in satisfactory signal production, in particular luciferase production, in the assay cells.

In certain embodiments, the assay cell is transfected with a cDNA containing ESR1 or the CYP19A1 gene of interest, typically a variant. The cDNA can be conveniently prepared using standard methodologies known to one skilled in the art. In certain embodiments, the cDNA can encode ER or AR wild type. In certain embodiments the cDNA can be a ER or AR variant. In further embodiments, the variants can contain one or more mutations different from the ER or AR wild type. In certain embodiments, the ESR1 or CYP19A1 variant contains one mutation. In certain embodiments, the specific variant may contain two mutations. In certain embodiments, the variant may contain three mutations. In certain embodiments the variant may contain four or more mutations.

In some embodiments, the AR or ER variant contains a missense mutation, insertion, or deletion. Examples of missense mutations in ER are L536P, V534E, 334InsC, E380R, and the like. An example of an insertion for ER is 344 Ins. Examples of missense mutations in AR are R192H, R435C, R435C, and the like.

In addition to the ESR1 or CYP19A1 cDNA encoding a variant, the linear expression cassette also comprises other components necessary or desirable for effective transfection. These components may vary depending on the particular assay cell chosen. Such other components typically include a promoter and terminator. Promoters include the cytomegalovirus (CMV) promoter, the SV40 promoter, elongation factor (EF)-1 promoter and the like. Typical terminators are SV40, hGH, BGH, and rbGlob. In addition a polyadenylation or poly(A) signal sequence is typically included. The assay cells can be either stably or transiently transfected with the linear expression cassette, but it typically is transiently transfected.

In some embodiments the assays cells are stably transfected with signal expression constructs. Stable transfection has the advantage of stability. In some embodiments the assay cells are transfected with signal expression constructs that have multiple copies of ERE, e.g., 2, 3, 4, 5, 6 or more copies. Usually multiple copies of ERE result in enhanced binding of ER to DNA. The introduced genetic materials are integrated into the host genome and sustain reporter gene expression even after assay cells replicate. The signal expression constructs also comprises other components necessary or desirable for effective stable transfection. These components may vary depending on the particular host cell chosen. Such other components can include, for example, a marker gene for selecting and identifying cells containing the signal expression constructs integrated into the host genome. Such marker gene can be, for example, genes that encode a fluorescent protein or encode antibiotic resistance such as resistance to hygomycin B, neomycin, puromycin and the like. The marker gene can be part of the signal expression construct or can be part of a separate construct co-transfected with the signal expression construct.

In another aspect, the present invention provides a method to determine whether a particular ESR1 or CYP19A1 variant is sensitive to treatment with a ER inhibitor or an AI, respectively, in a cell. The method involves preparing a cDNA containing an ESR1 or CYP19A1 variant of interest followed by transfecting the cDNA into an assay cell. In certain embodiments, the transfected cells are then exposed to an inhibitor. Inhibitors of ER include raloxifene, tamoxifen, toremifene, fulvestrant, and the like. Inhibitors of AR include anastrozole, exemestane, letrozole, and the like. A convenient approach is to obtain a concentration dependent response for an inhibitor by performing a dose dependent curve study. By way of example, fulvestrant can be used from about 0.01 nM to about 10 nM, in one embodiment from about 0.05 nM to about 5 nM, in another embodiment from about 0.1 nM to about 1 nM. The other inhibitors can be used at the same concentrations as fulvestrant or modified as appropriate. The sensitivity of the ER variant toward a particular inhibitor can be conveniently measured by an increasing light emission as compared to a negative control (i.e., an assay cell exposed to vehicle alone without the inhibitor).

In certain embodiments, the cells express a knock down or knockout of endogenous ESR1 or CYP19A1. In certain embodiments, the knock down is a genomic modification of at least a portion of the ESR1 gene or the CYP19A1 gene. In certain embodiments, the genomic modification is performed using CRISPR-CAS9 technology. In certain embodiments, the genomic modification is performed using TALENs or recombination technology.

In one aspect, the present invention provides an assay to test patient variants of the ESR1 or CYP19A1 gene, as identified by next generation sequencing (NGS), thus determining potentially hyperactive and/or inhibitor resistant mutations. ER acts as a transaction activator. When cells are treated with a ER inhibitor and ER is inhibited, transcriptional activation of ER target genes is prevented which results in decreasing the growth and replication of the cell, thereby inhibiting cancer cells. Similarly, when AR is inhibited, there is less conversion of androgen, e.g., testosterone, to estrogen resulting is less estrogen being available to react with ER downstream.

In certain embodiments, the present assay may be used to determine whether a patient ER or AR variant will respond to a specific inhibitor. The specific variant is determined from a patient's biological sample. The method involves preparing a cDNA containing a ER or AR variant from a patient followed by transfecting the cDNA into a cell. Depending upon the particular cell line and other conditions, if any endogenous AR and/or ER can be produced by the assay cells, the cells can undergo a genomic modification for gene deletion (knockout) or knockdown to reduce or prevent interference with the assays of the invention. In another embodiment, if the assay cells do not express the endogenous AR or ER gene, then no such genomic modification will be needed. After determining the activity of the ER and/or AR variant of a patient, the physician can use this information to adjust therapy. Appropriate therapy may include surgery, radiation, chemotherapy, immunotherapy, hormone therapy, targeted therapy, and the like. For example, if the ER variant is constitutively active and its activity is independent of estrogen, then AR inhibitors should not be used. If the ER variant activity depends on estrogen, then AR inhibitors should be used.

In some embodiments, the biological sample is from patients selected from: blood, serum, and tumor tissue. The biological sample can be tissue or cells from a breast tumor. In some embodiments, the biological sample may be freshly isolated. In some embodiments, the biological sample may be frozen. In some embodiments, the biological sample may be fixed. The biological sample can be processed to obtain ER and/or AR genomic information and the AR/ER variants can be sequenced using know techniques, e.g., NGS. The genomic information can then be used to generate polynucleotides that can then be used to transfect the assay cells. In an alternate embodiment, the AR/ER polynucleotides can be isolated directly from the biological sample and used to transfect the assay cells.

The assay cells of the invention are capable of expressing a reporter regulated by ERE. In some embodiments, the test cell is a eukaryotic cell. In some embodiments the assay cells are mammalian cells, such as rat, mouse, hamster, monkey and human cells. In some embodiments, the test cell may be a primary cell or a cell line. In another embodiment, an assay cell is a non-cancerous cell. In another embodiment, an assay cell is derived from a cell line. In another embodiment, an assay cell is amenable by transfection. In another embodiment, an assay cell is amenable by transient transfection. In another embodiment, an assay cell is a cell in which the expression of one or more endogenous genes have been reduced or eliminated by any molecular method. For example, in some embodiments it may be desirable to knockdown or knockout endogenous ESR1 and/or CYP19A1. Specific examples of cells useful in the assays of the invention include HEK293 (human embryo kidney), MCF-7 (human breast cancer), Hela (human cervix epithelial carcinoma), HT29 (human colon adenocarcinoma grade II), A431 (human squamous carcinoma), IMR 32 (human neuroblastoma), K562 (human chronic myelogenous leukemia), U937 (human histiocytic lymphoma), MDA-MB-231 (Human breast adenocarcinoma), SK-N-BE(2) (human neuroblastoma), SH-SY5Y (human neuroblastoma), HL60 (human promyelocytic leukemia), CHO (hamster Chinese ovary), COS-7 (monkey African green kidney, SV40 transformed), S49 (mouse lymphoma), Ltk (mouse C34/connective tissue), NG108-15 (mouse neuroblastoma×Rat glioma hybrid), B35 (rat nervous tissue neuronal), B50 (rat nervous tissue neuronal), B104 (rat nervous tissue neuronal), C6 (rat glial tumor), Jurkat (human leukemic T cell lymphoblast), BHK (hamster Syrian kidney), Neuro-2a (mouse albino neuroblastoma), NIH/3T3 (mouse embryo fibroblast), A549 (human adenocarcinoma alveolar epithelial), Be2C (human neuroblastoma), SW480 (human lymph node metastasis), Caco2 (human epithelial colorectal adenocarcinoma), THP1 (human acute monocyte leukemia), IMR90 (human lung fibroblast), HT1080 (human fibrosarcoma), LnCap (human prostate adenocarcinoma), HepG2 (human liver carcinoma) PC12 (rat pheochromocytoma), or SKBR3 (human breast cancer) cells. In another embodiment, an assay cell is U20S cell. In another embodiment, an assay cell is NCI60 cell lines, such as, A549, EKVX, T47D, HT29.

In the assays using AR or ER inhibitors, the inhibitors that are exposed to the assay cells are typically solubilized or suspended in a vehicle. A control is typically performed with vehicle without the inhibitor. Depending on the compound to be utilized as an inhibitor in the assay, suitable vehicles include dimethylsulfoxide (“DMSO”), dimethylformamide (“DMF”), water, aliphatic alcohols, and mixtures thereof.

In some embodiments, the patient has been diagnosed positive for cancer. In some embodiments, the patient is subjected to targeted therapy treatment regimen with known or unknown treatment results. In some embodiments, the patient has an available patient tumor molecular profiling (IHC, FISH, PCR and sequencing). In some embodiments, the patient has available patient history as well as outcome (patient response, resistance, recurrence and survival rates).

According to some embodiments, there is provided a kit for determining AR or ER activity in a patient or for determining a patient's response to estrogen inhibitors or AIs. In some embodiments, there is provided a kit for assessing patient specific mutations.

In some embodiments, the invention provides a kit for determining the molecular cancer profile in a subject, by identifying patient specific AR or ER variants. In another embodiment, the kit comprises at least one means of detecting a reporter gene. In some embodiments, the kit contains one or more of: a substrate or container for holding nucleic acid molecules and/or test cells, directions for carrying out the assay(s), test cells, transfection reagents, or any combination thereof.

Compositions of the present invention may, if desired, be presented in an article of manufacture, which may contain diagnostic reagents and printed instructions for use. The kit may also be accommodated by a notice associated with the container in a form prescribed by a governmental agency regulating the manufacture, use or sale of assay products.

The assays of the invention are carried out under culture conditions effective for protein expression from cells. In some embodiments, effective culture conditions include, but are not limited to, effective media, bioreactor, temperature, pressure, nutrients, pH and oxygen conditions that permit protein expression. In one embodiment, an effective medium refers to any medium in which a cell is cultured to produce the recombinant polypeptide of the present invention. In some embodiments, a medium typically includes an aqueous solution having assimilable carbon, nitrogen and phosphate sources, and appropriate salts, minerals, metals and other nutrients, such as vitamins. In some embodiments, cells of the present invention can be cultured in conventional fermentation bioreactors, shake flasks, test tubes, microtiter dishes and petri plates. In some embodiments, culturing is carried out at a temperature, pH and oxygen content appropriate for a recombinant cell. In some embodiments, culturing conditions are within the expertise of one of ordinary skill in the art.

The assays are performed in a vessel capable of holding the cells and reagents and not interfering with assay results. In some embodiments the assay cells adhere to the surfaces of the vessel, e.g., the surfaces of assay plate wells. In some embodiments the assays are miniaturized and use multi-well plates known in the art. In certain embodiments, the present assay can be conveniently used in a 96 well plate, but can also be adopted for high throughput in 384 well plates or 1536 well plates. One skilled in the art will be able to easily optimize the well plates to suit throughput necessity. Plates for the assays are typically made of a polymer, e.g., polystyrene and the like. In some embodiments the plates are surface treated to facilitate adherence of the assay cells to the wells of the plate, such treatment is commonly referred to as “tissue culture treated”. The surface treatment is typically an oxygen plasma discharge that renders the surface of the wells more hydrophilic. In some embodiments dispensing the cells and/or reagents for the assays into the wells of the plates is automated. In some embodiments the cells and/or reagents are dispensed continuously at a high speed. In one embodiment an acoustic liquid dispenser is used to dispense the reagents, e.g., estrogen, ER inhibitors, testosterone, and AR inhibitors.

Any discussion of the content of references cited herein is intended merely to provide a general summary of assertions made by the authors of the references, and does not constitute an admission as to the accuracy of the content of such references. The following examples are provided to further illustrate various preferred embodiments and techniques of the invention. It should be understood, however, that these examples do not limit the scope of the invention described in the claims. Many variations and modifications are intended to be encompassed within the spirit and scope of the invention

### EXAMPLES

### Example 1 Next-Generation Sequencing to Interrogate Mutation of Tumor

Genetic change, mutation of genes, is one of the mechanisms to acquire resistance in breast cancer during hormonal therapy. To survey the mutation status of estrogen receptor (ESR1) and aromatase (CYP19A1) genes, whole-exome sequencing of the tumor was performed. Cancer tissue was isolated from formalin fixed paraffin embedded (FFPE) blocks of tumor. Genomic DNA was extracted and was used to prepare a library for next-generation sequencing. The identified mutations, which caused change in amino acid sequence, were picked to examine its functional effect on the genes in our proprietary cell-based assay described below. The patient gene carrying the identified mutation is constructed using PCR mediated overlapping extension in a format of linear expression cassette.

### Example 2 Construction of Linear Expression Cassette of Human Estrogen Receptor (ESR1)

In order to study the effect of unknown mutations in human ESR1 gene, we decided to generate linear expression cassette, which contained CMV promoter controlling expression of ESR1 coding sequence followed by terminator and polyadenylation signal. To do so, overlapping extension PCR was employed to construct the linear expression cassette using expression plasmid of human ESR1 as PCR template. Using this method, the construction of linear expression cassette takes around 4-8 hours. However, the traditional cloning method to generate expression plasmid takes around 2-4 days. Therefore, making patient gene in linear expression cassette format is highly advantageous for a clinical diagnostic test because of its quick turn-around time.

### a) Construction of Expression Plasmid of Human ESR1

cDNA plasmid encoding human ESR1 gene was purchased (Dharmacon). We amplified the coding sequence of human ESR1 gene by the PCR. We inserted NheI and XhoI restriction enzyme sites to forward and reverse primers respectively for purpose of cloning. The PCR products containing the coding sequence of human ESR1 were sub-cloned into the pcDNA3.1 (+) using NheI and XhoI restriction enzymes. The nucleotide sequence of human ESR1 was verified by the DNA sequencing. This human ESR1 expression plasmid was used as PCR template to construct the linear expression cassettes of wild-type ESR1 or mutated forms of ESR1.

### b) Generation of Linear Expression Cassette of Human Normal and Mutated ESR1

A linear expression cassette of human wild-type ESR1 was generated by UF-CMV forward and BGH-UR reverse primers. The amplified products were gel-purified. The DNA concentration was quantitated by the optical density at 260 nm using Nanodrop.

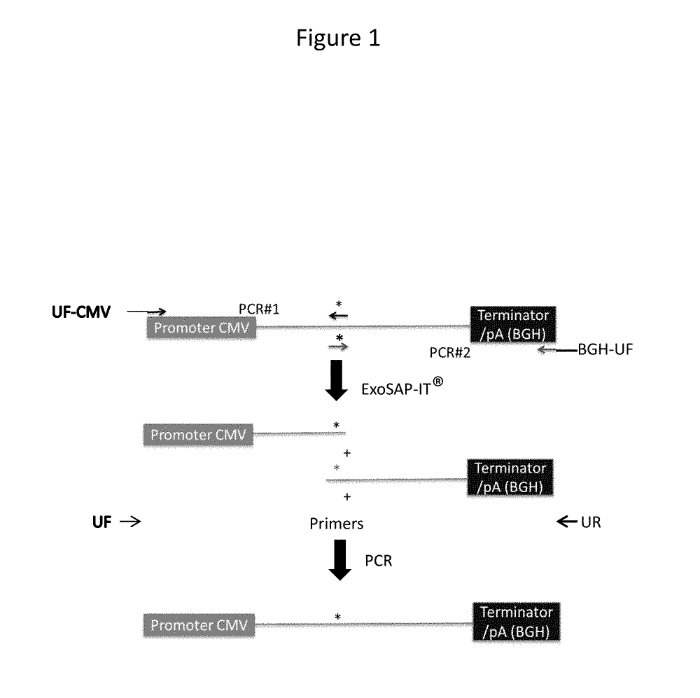

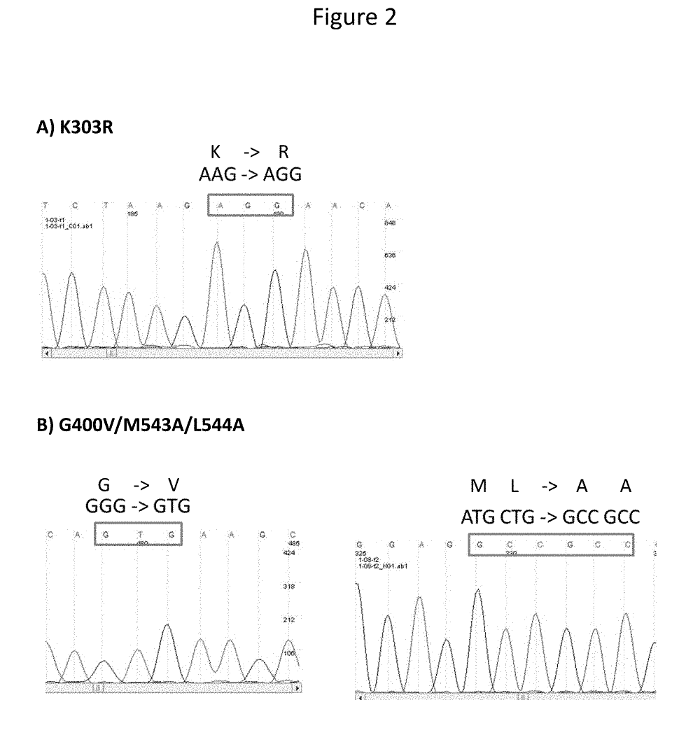

A linear expression cassette of mutated ESR1 was generated by PCR mediated overlapping extension method. A pair of forward and reverse primers containing the targeted mutations was designed. The mutated codon (3 nucleotides) was located in the middle of primer flanked by 18 nucleotides on each side. Two separate PCR, named as PCR#1 and PCR#2 in FIG. 1, were performed using UF-CMV forward and mutated reverse primers, or mutated forward and BGH-UR primers. The PCR products were cleaned up by ExoSAP-IT® (Affymetrix) to remove unconsumed dNTPs and primers. These two treated PCR products were mixed together followed by dilution with water. We performed second round of PCR using the diluted PCR mixture as template, and UF and UR primers (FIG. 1). The amplified products were gel-purified. The DNA concentration was quantitated by the optical density at 260 nm using Nanodrop. The targeted mutations were successfully incorporated in the ESR1 gene during the PCR mediated overlapping extension method. The introduced mutations were confirmed by the DNA sequencing (FIG. 2). For K303R mutant, codon “AAG” encoding lysine at amino acid #303 was changed into codon “AGG” encoding arginine (FIG. 2A). Same observation was found when we sequenced the triple mutant, G400V/M543A/L544A (FIG. 2B).

### Example 3 Identification of Transfection Method

HEK 293 cells are a specific cell line originally derived from human embryonic kidney cells grown in tissue culture. HEK 293 cells have been widely used in cell biology research for many years, because of their reliable growth and propensity for transfection. In general, there are two major types of transfection, forward and reverse.

The most routinely employed transfection protocol where cells were seeded a day prior to transfection was referred to as “forward transfection”. Forward transfection methods worked well for most adherent cell types that were seeded a day prior to transfection in order to achieve an actively dividing cell population at the time of transfection. A “reverse transfection” protocol where freshly passaged cells were added to transfection complexes is ideal as it reduced hands-on time for the end user. In this scenario, cells were not adhered to the plate surface by the time they interacted with the transfection complexes.

### a) Comparison of Forward or Reverse Transfection Protocols

In order to test the possibility of using a “reverse transfection” method to reduce total assay time (about a day), a reporter plasmid, CDH1-luc, containing active CDH1 promoter was used. Since our clinical assay employed the linear expression cassette to express the patient ESR1 or CYP19A1gene which contains mutation found in the tumor, we then performed PCR to generate linear version of CDH1-luc. This linear reporter was gel-purified. The DNA concentration was quantitated by the optical density at 260 nm using Nanodrop.

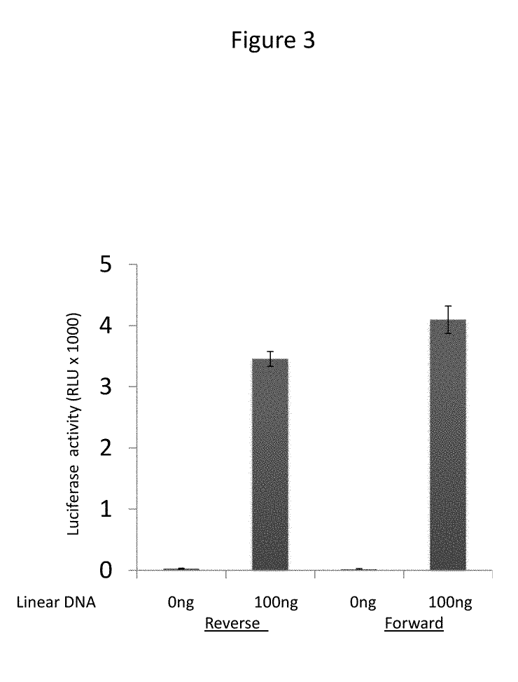

To compare the “forward transfection” and “reverse transfection” protocols, we transiently transfected HEK 293 cells with 100 ng of purified linear reporter using TransIT-293 transfection reagent (Minis Bio). This transfection reagent was optimized to give maximum transfection performance in HEK 293 cells. The transfection efficiency was measured by Nano-Glo® Luciferase assay (Promega) 24 hours post-transfection (FIG. 3). We detected significant luciferase activity over the background signal using either forward or reverse transfection protocol. There is no substantial difference of luciferase signal between these two protocols. Therefore, these results show that “reverse transfection” protocol can be used to transiently transfect linear DNA into HEK 293 cells.

### b) Expression of Wild-Type and Mutated ESR1 in HEK 293 Cells Using Linear Expression Cassette and “Reverse Transfection” Protocol

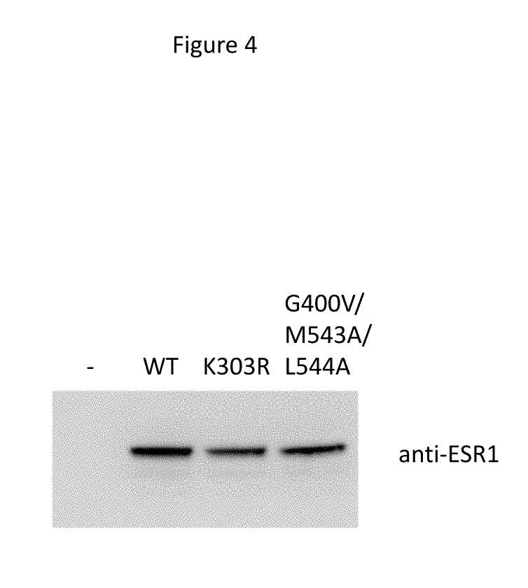

In order to confirm that a linear expression cassette was able to express estrogen receptor proteins, we performed a “reverse transfection” protocol to transiently transfect the purified linear expression cassettes of wild-type, K303R and triple mutations (G400V/M543A/L544A) of ESR1 into the HEK 293 cells. Transfected cells were lysed 48 hours post-transfection and cellular lysates were prepared. We performed an immunoblot assay using anti-ESR1 to detect the expression of ESR1. We detected expression of ESR1 when HEK 293 cells were transfected with linear expression cassettes of ESR1 (FIG. 4).

### Example 4 Identification of Reporter System to Measure ESR1 Activity

Estrogen receptors (ERs) are receptors that are activated by the hormone estrogen. It also belongs to DNA-binding transcription factor. In resting cells, the estrogen receptors reside in the cytoplasm. Upon activation by estrogen, the estrogen receptor translocates into the nucleus and binds to DNA to activate transcription of its target genes. ER binds to specific DNA sequences called estrogen response elements (EREs) with high affinity. The consensus ERE DNA sequence has been reported and delineated.

### a) Construction of 3×ERE-Luc Reporter Plasmid

Estrogen receptor is a DNA-binding transcription factor and its consensus ERE DNA sequence, GTCAGGTCACAGTGACCTGAT (SEQ ID NO: 93), has been reported. We designed an oligonucleotide containing three copies of ERE sequence, which was then subcloned into pNL3.2 luciferase reporter plasmid. Three copies of ERE sequence in the constructed plasmid, 3×ERE-luc, were confirmed by DNA sequencing.

### b) Validation of 3×ERE-Luc Reporter by Overexpression of Estrogen Receptor

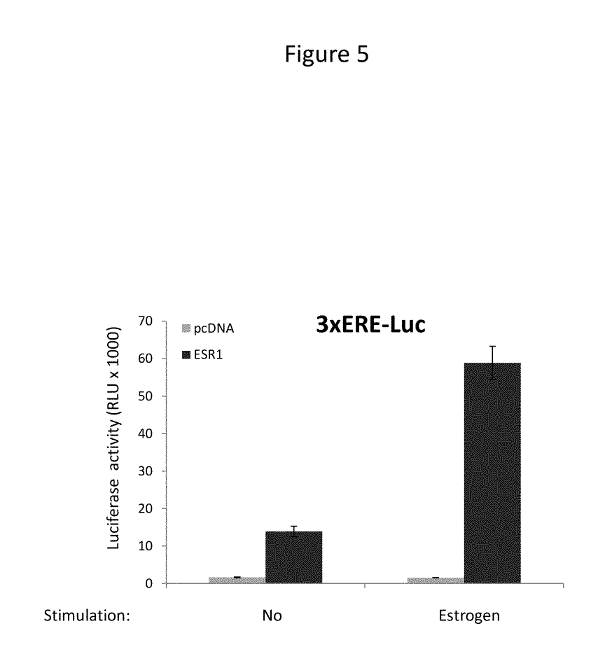

In order to examine whether 3×ERE-luc is capable of measuring the activity of estrogen receptor, we transfected HEK 293 cells with either 3×ERE-luc and pcDNA or 3×ERE-luc and ESR1 expression plasmid (FIG. 5). After 24 hours, the transfected cells were exposed to estrogen for 6 hours. The luciferase reporter activity was measured by Nano-Glo® Luciferase assay (Promega). In the absence of ESR1 expression plasmid, no reporter activity was detected even though the cells were exposed to estrogen. It strongly indicated that HEK 293 cells did not express estrogen receptor. However, when estrogen receptor was overexpressed, we detected a significant increase of luciferase reporter activity upon estrogen exposure. This observation shows that our 3×ERE-luc reporter can be used to measure the estrogen receptor activity.

### c) Validation of 3×ERE-Luc Reporter by Inhibitors of Estrogen Receptor

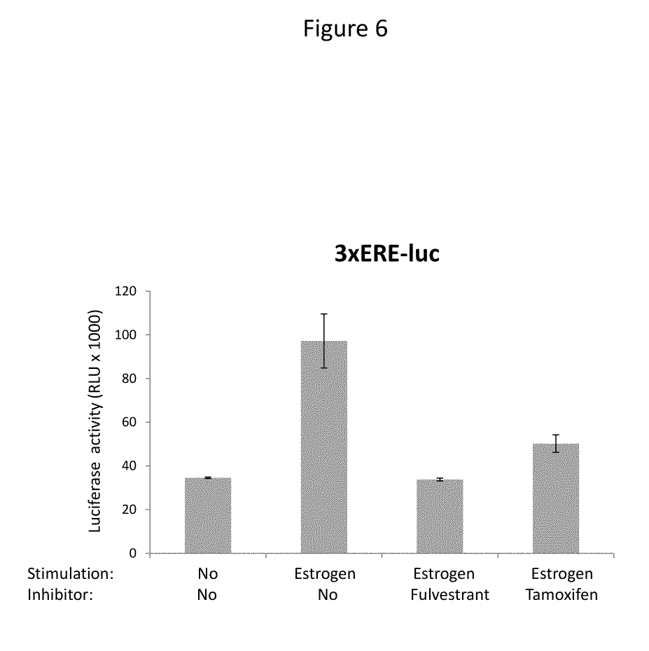

To further validate the 3×ERE-luc reporter system, we co-transfected HEK 293 cells with 3×ERE-luc and ESR1 expression plasmid. After 24 hours, the transfected cells were exposed to estrogen in the presence or absence of estrogen inhibitors, fulvestrant and tamoxifen, for 6 hours (FIG. 6). In the absence of inhibitors of ER, we detected high luciferase reporter activity upon estrogen exposure. Such increase of reporter activity was reduced by treating the transfected cells with inhibitors of ER, such as fulvestrant and tamoxifen. This observation shows that 3×ERE-luc reporter system can be used to measure the response of estrogen receptor toward inhibitors.

### d) Validation of 3×ERE-Luc Reporter by Activating Mutation, Y537S, of Estrogen Receptor

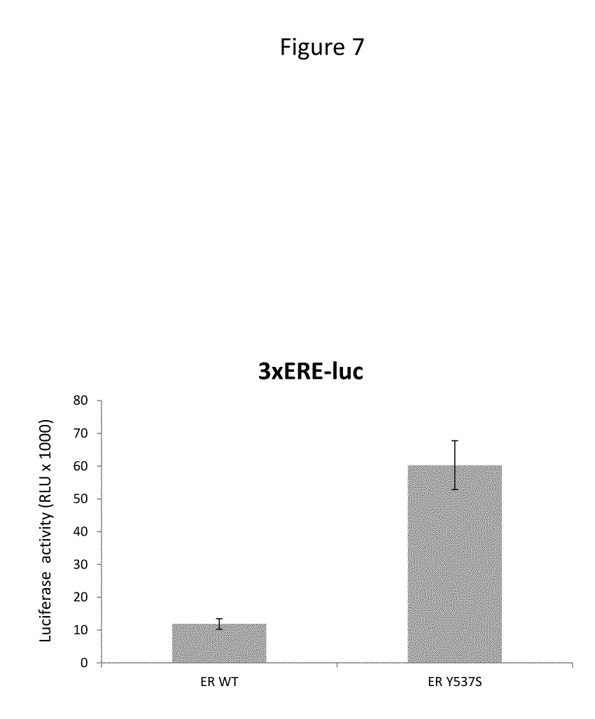

In addition to validating the assay by overexpressing estrogen receptor and using inhibitors of ER, we further evaluated the 3×ERE-luc reporter system using activating mutation, Y537S, of estrogen receptor. This mutation activates estrogen receptor even in the absence of estrogen. To test our reporter system, we co-transfected 3×ERE-luc reporter plasmid with either wild-type or Y537S ESR1 expression plasmid into HEK 293 cells. The luciferase reporter activity was measured 24 hours post-transfection. The transfected cells expressing Y537S ER showed increased luciferase reporter activity compared to cells expressing wild-type ER (FIG. 7). This result shows that that 3×ERE-luc reporter system can be used to measure the effect of mutation on activity of estrogen receptor.

### Example 5 Construction and Characterization of Stable Reporter Cells Containing 3×ERE-luc Reporter Plasmid

The 3×ERE-luc reporter system has been validated by multiple approaches. This reporter system can be used to measure the estrogen receptor activity, its response toward the inhibitors and the mutation effect on activity of estrogen receptor. In order to use this reporter system in a practical clinical diagnostic test, we generated reporter cells, in which 3×ERE-luc plasmid was stably integrated in the genome of HEK 293 cells.

### a) Selection of Stable Cell Clones Carrying 3×ERE-Luc Plasmid

Since 3×ERE-luc plasmid did not contain selectable marker, we co-transfected 3×ERE-luc with pIRES-hygB, which contained hygomycin B resistance gene, in 5:1 ratio into HEK 293 cells. Antibiotic, hygromycin B, was added to the culture medium 48 hours post-transfection. Any cells survived through the selection process suggested that those cells not only expressed hygomycin B resistance gene, which was encoded in pIRES-hygB plasmid, but also carried 3×ERE-luc reporter plasmid as we co-transfected 3×ERE-luc with pIRES-hygB in 5:1 ratio.

b) Characterization of Single Cell Clones after Hygomycin B Selection

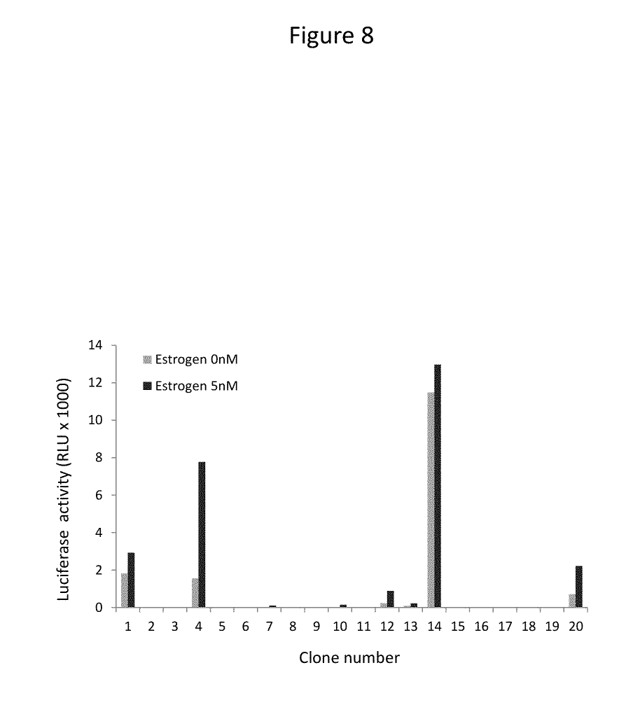

Twenty single cell clones were picked from the selection plate and were expanded. In order to confirm the integration of 3×ERE-luc reporter plasmid, these single cell clones were transfected with ESR1 expression plasmid. After 24 hours, the transfected cells were exposed to estrogen for 6 hours. The luciferase reporter activity was measured by Nano-Glo® Luciferase assay (Promega) (FIG. 8). Six out of twenty clones showed detectable basal luciferase reporter activity in the absence of estrogen. Upon exposure to estrogen, 8 out of twenty clones showed detectable luciferase reporter activity. Among these 8 clones, clone #4, #12 and #20 showed the best fold of induction. Therefore, these three clones were used for further analysis.

### c) Dose Response Curve of Estrogen Using Clone #4, #12 and #20

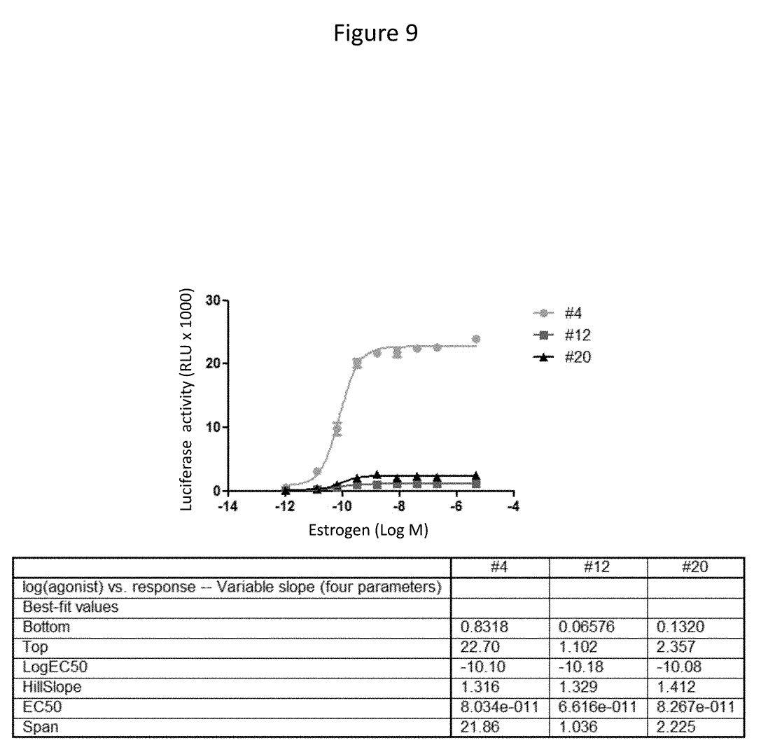

3×ERE-luc single cell clone #4, #12 and #20 were transfected with ESR1 expression plasmid. After 24 hours, the transfected cells were exposed to a serial dilution of estrogen for 6 hours. The luciferase reporter activity was measured. All clones responded to estrogen exposure in a dose dependent manner with similar sensitivity (FIG. 9). 3×ERE-luc clone #4 gave the best response with good background to signal ratio. Therefore, this clone was selected for further characterization in our assay development.

### d) Inhibition Curve of Clone #4 Using Fulvestrant

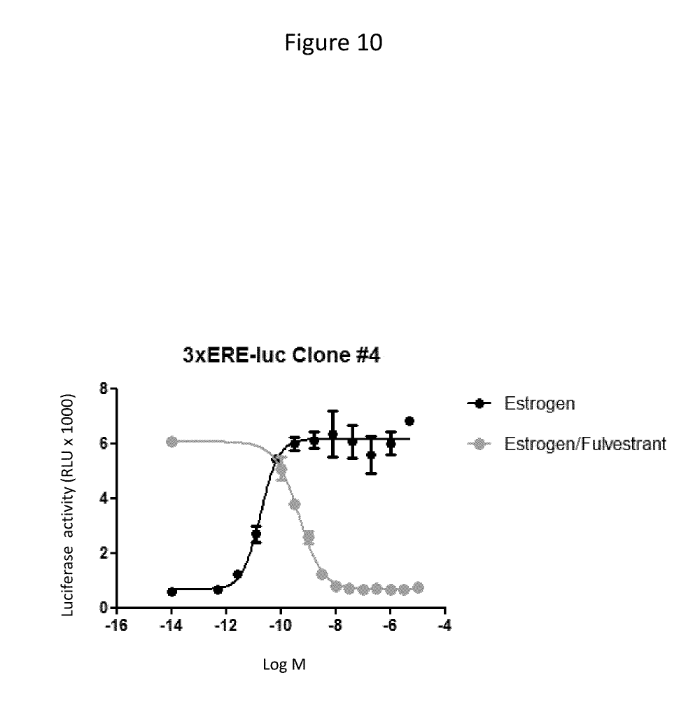

Clone #4 showed dose dependent response toward estrogen exposure. We decided to further characterize this clone by examining its response toward inhibitor treatment in the presence of estrogen. To do so, 3×ERE-luc single cell clone #4 was transfected with ESR1 expression plasmid. After 24 hours, the transfected cells were treated with a serial dilution of estrogen or serial dilution of fulvestrant in the presence of 64 pM of estrogen for 6 hours. The luciferase reporter activity was measured. This inhibitor was able to block activation of the reporter by estrogen in a dose dependent manner (FIG. 10). This observation confirmed that the 3×ERE-luc reporter plasmid was successfully integrated in the genome of this clone #4. This integrated reporter also retained the ability to measure the activity of estrogen receptor and its response toward the inhibitors.

### e) Linear Expression Cassette of Wild-Type Estrogen Receptor

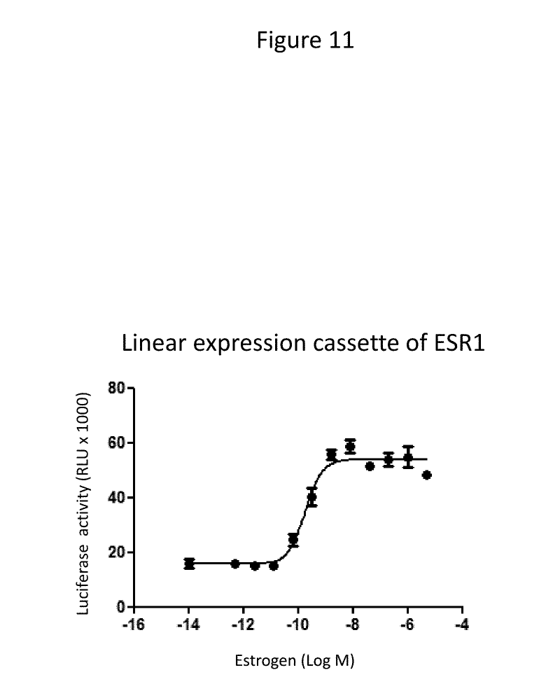

In the clinical assay of the invention, expression of patient estrogen receptor is in the linear expression cassette format instead of plasmid DNA. Therefore, we transfected clone #4 with linear expression cassette of wild-type ESR1. After 24 hours, the transfected cells were treated with a serial dilution of estrogen for 6 hours. The luciferase reporter activity was measured. The cells transfected with linear DNA showed dose dependent response to estrogen exposure similar to the cells transfected with expression plasmid of ESR1 (FIG. 11). This observation demonstrated that linear expression cassette can be used to produce functional estrogen receptor.

### Example 6 Optimization of Cell-Based Assay for Estrogen Receptor Using 3×ERE-Luc Clone #4

In example 3, we successfully generated stable cell clones containing 3×ERE-luc reporter plasmid. Among them, clone #4 showed the best response toward estrogen exposure. In order to use this clone #4 as a clinical assay, we performed optimization such as DNA amount, culture duration of clone #4 and treatment methodology.

### a) DNA Amount of Linear Expression Cassette of ESR1

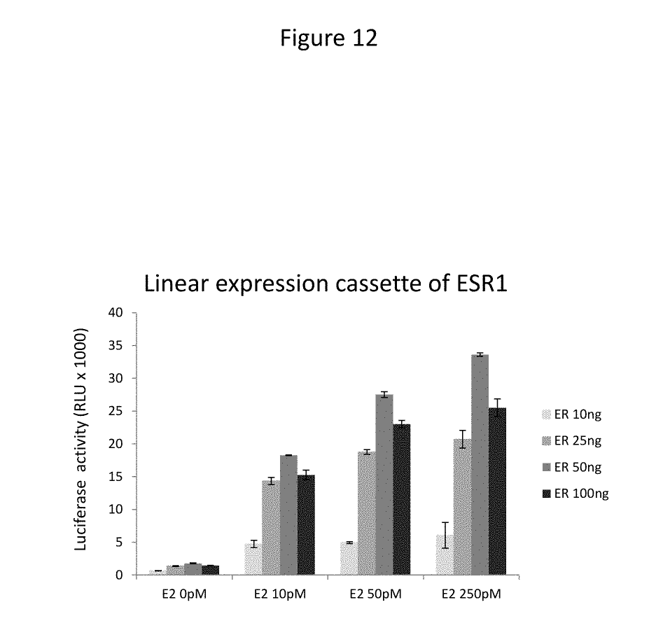

We first optimized the amount of linear DNA used for transfection. The cells from clone #4 were transfected with 10 ng, 25 ng, 50 ng and 100 ng of linear expression cassette of ESR1. After 24 hours, the transfected cells were treated with different concentration of estrogen for 6 hours as shown in FIG. 12. The luciferase reporter activity was measured. The cells transfected with linear DNA showed dose dependent response to estrogen exposure in all tested conditions except 10 mg of linear expression cassette of ESR1. Among them, the cells transfected with 50 ng of linear expression cassette of ESR1 gave the best activation profile upon estrogen treatment. Therefore, 50 ng of linear DNA was used in the assay for further evaluation.

b) Exposure Methodology—2, 4 and 6 Hours Verse all-in-One

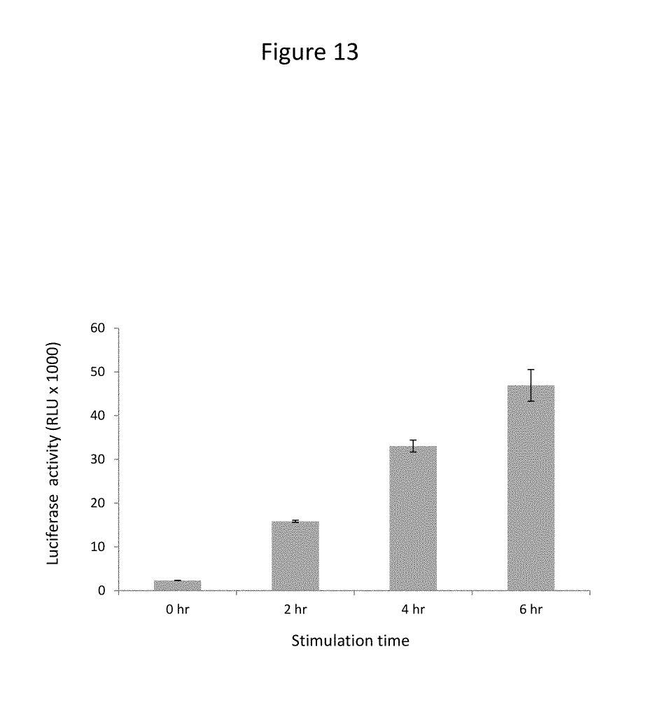

In our previous experiments, the cells were first transfected with either ESR1 expression plasmid or linear expression cassette of ESR1. Estrogen was added to the cells 24 hours post-transfection. Reporter assay was performed 6 hours after the exposure. In order to examine whether we can reduce the exposure time, same experiment was performed except the transfected cells were exposed to estrogen for 2, 4, or 6 hours. In FIG. 13, the result clearly showed that exposure time can be shortened but luciferase reporter activity was compromised.

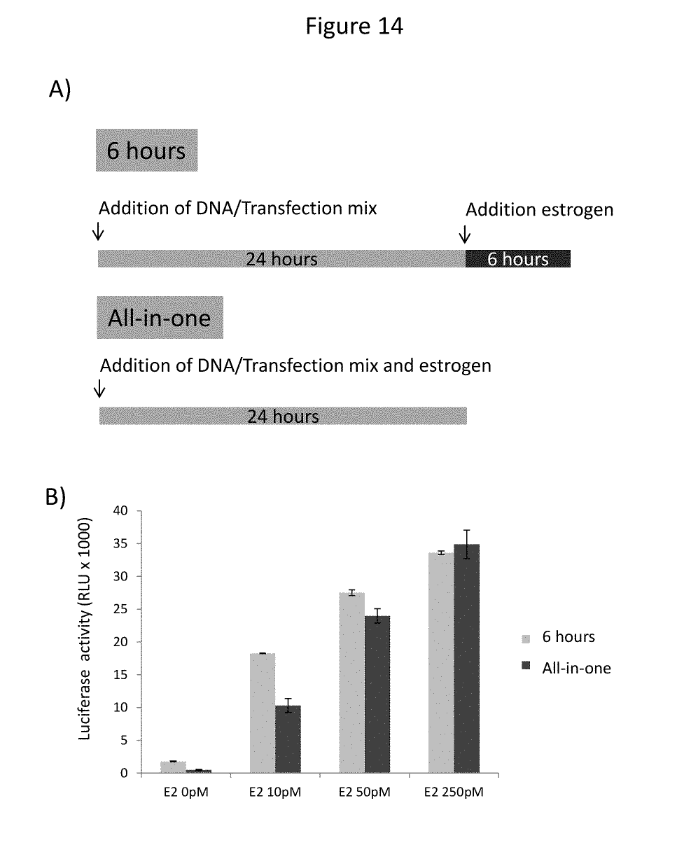

To further explore other possibility to reduce the assay turnaround time, we compared two different exposure methods. The first one was addition of estrogen to the cells 24 hours post-transfection for 6 hours. The other method was treating the cells with estrogen when we added the DNA transfection complex to the cells. The reporter activity was measured 24 hours post-transfection. The second method was named as “all-in-one”. The comparison of these two methods was illustrated in FIG. 14A. FIG. 14B showed that all-in-one method gave a good dose response toward estrogen exposure. Although the all-in-one method showed reduced activation at 10 pM estrogen compared to the 6 hours treatment method, the new all-in-one method reduced total time required by 6 hours. Therefore, the results demonstrated that the all-in-one method is satisfactory in the estrogen receptor assays of the invention.

### c) Culturing Condition of 3×ERE-Luc Clone #4

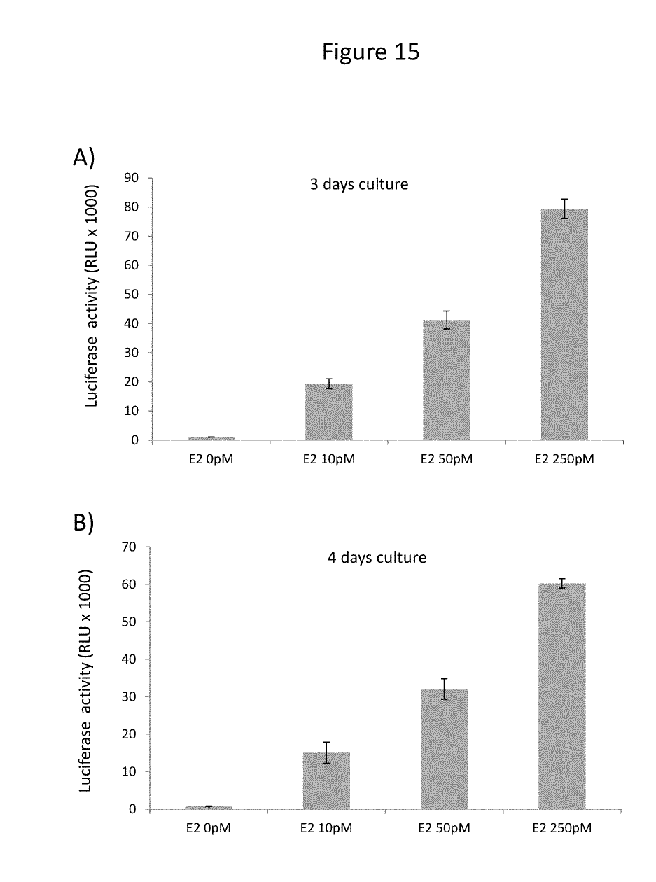

Consistency and reproducibility are important for a practical clinical assay. In order achieve this, we standardized the method to culture 3×ERE-luc clone #4 cells. We compared two different culture conditions. The cells were cultured in 10 cm tissue culture plate seeded with either 0.75×10̂6 cells for 4 days (4 days culture) or 1.5×10̂6 cells for 3 days (3 days culture). The same exposure experiment was performed using the cells cultured in these two different conditions. In FIG. 15, the result demonstrated that both culture methods gave similar exposure profiles. Therefore, the clone #4 cells were cultured in either condition prior to transfection with linear expression cassette of ESR1.

### Example 7 Partial Agonist of Estrogen Inhibitors, Tamoxifen, Toremifene and Raloxifene

Selective estrogen receptor modulators (SERMs) such as tamoxifen, toremifene and raloxifene, are effective in the treatment of many estrogen receptor-positive breast cancers and have also proven to be effective in the prevention of breast cancer in women at high risk for the disease. SERMs are characterized by their diverse range of agonist/antagonist actions on estrogen receptor (ER)-mediated processes. They have the ability to act as either ER antagonists by blocking estrogen action through its receptor, as ER agonists by displaying estrogen-like actions, or as ER partial agonists/antagonists with mixed activity. Frequently, these differences in SERM activity depend upon the target gene promoter, as well as the cell or tissue background. Therefore, we examined these special properties of SERMs in our 3×ERE-luc clone #4 reporter assay cells.

### a) Tamoxifen

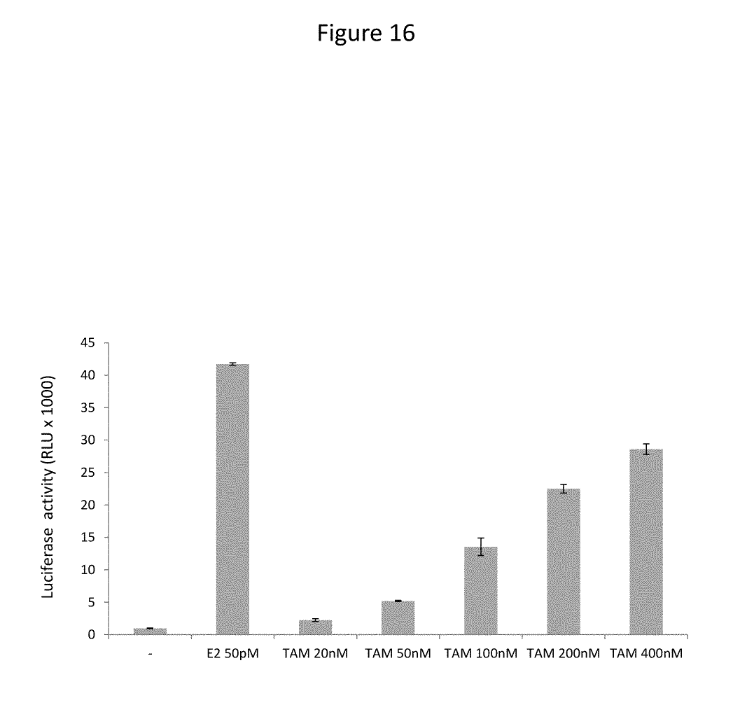

In order to examine the estrogen-like action of tamoxifen, we transfected an expression plasmid of estrogen receptor into the 3×ERE-luc clone #4 reporter cells. The cells were treated with 50 pM of estrogen or a serial dilution of tamoxifen. The reporter activity was measured 24 hours post-transfection (FIG. 16). The cells responded to 50 pM of estrogen and gave a robust induction of luciferase reporter activity. The reporter activity also increased in a dose dependent manner upon tamoxifen treatment. This observation demonstrated that the estrogen-like activity of tamoxifen existed in our reporter assay cell lines.

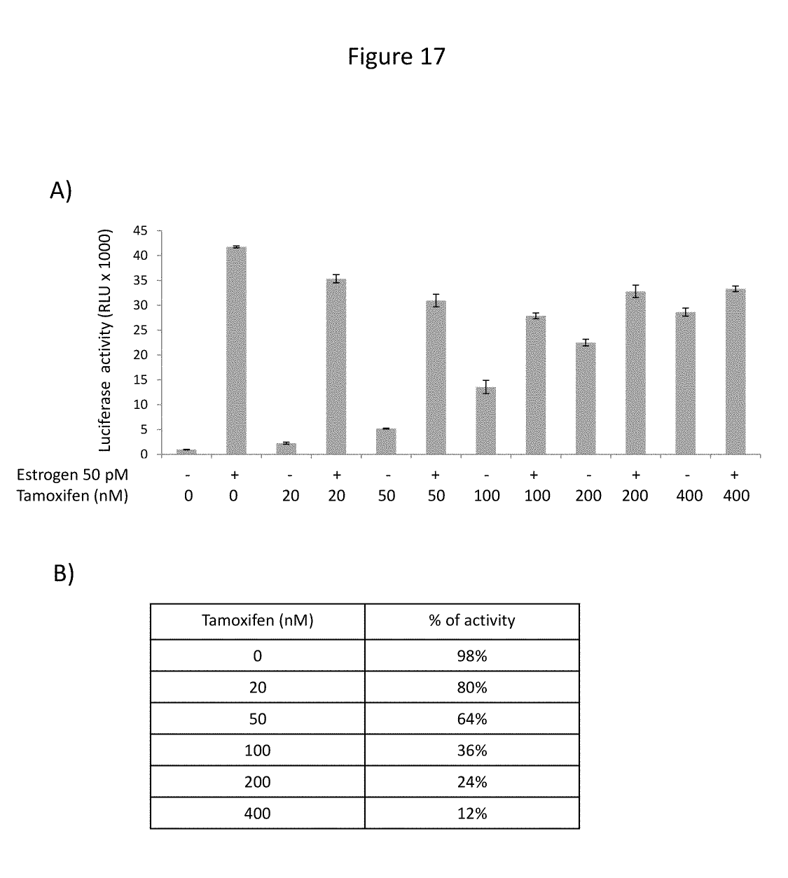

In order to measure the inhibitory activity of tamoxifen, we transfected an expression plasmid of estrogen receptor into the 3×ERE-luc clone #4 reporter assay cells. The cells were treated with a serial dilution of tamoxifen in the presence or absence of 50 pM of estrogen. The reporter activity was measured 24 hours post-transfection (FIG. 17A). To calculate the activity of estrogen receptor exposed to estrogen in the presence of tamoxifen, we used the following formula;

Activity of estrogen receptor=[(Luciferase activity of cells treated with estrogen in the presence of tamoxifen−Luciferase activity of cells treated with tamoxifen)/(Luciferase activity of cells treated with estrogen−Luciferase activity of cells without treatment)]×100%

In the FIG. 17B, the data clearly show that the activity of estrogen receptor decreased as the concentration of tamoxifen increased. Therefore, we demonstrated that the activity of estrogen receptor can be calculated from the luciferase reporter assay using the above stated formula.

### b) Toremifene

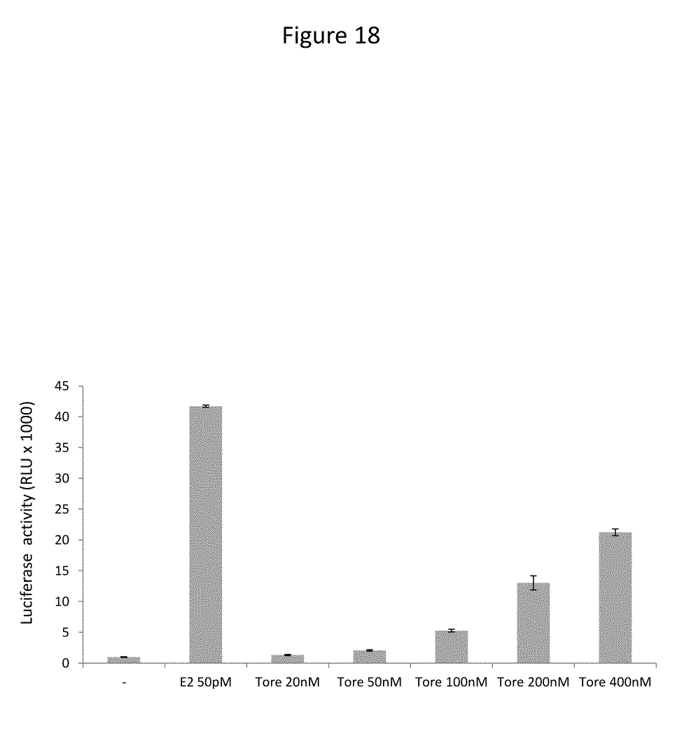

In order to examine the estrogen-like action of toremifene, we transfected an expression plasmid of estrogen receptor into the 3×ERE-luc clone #4 reporter assay cells. The cells were treated with 50 pM of estrogen or a serial dilution of toremifene. The reporter activity was measured 24 hours post-transfection (FIG. 18). The cells responded to 50 pM of estrogen and gave a robust induction of luciferase reporter activity. The reporter activity also increased in a dose dependent manner upon toremifene treatment. This observation demonstrated that the estrogen-like activity of toremifene existed in our reporter cell lines.

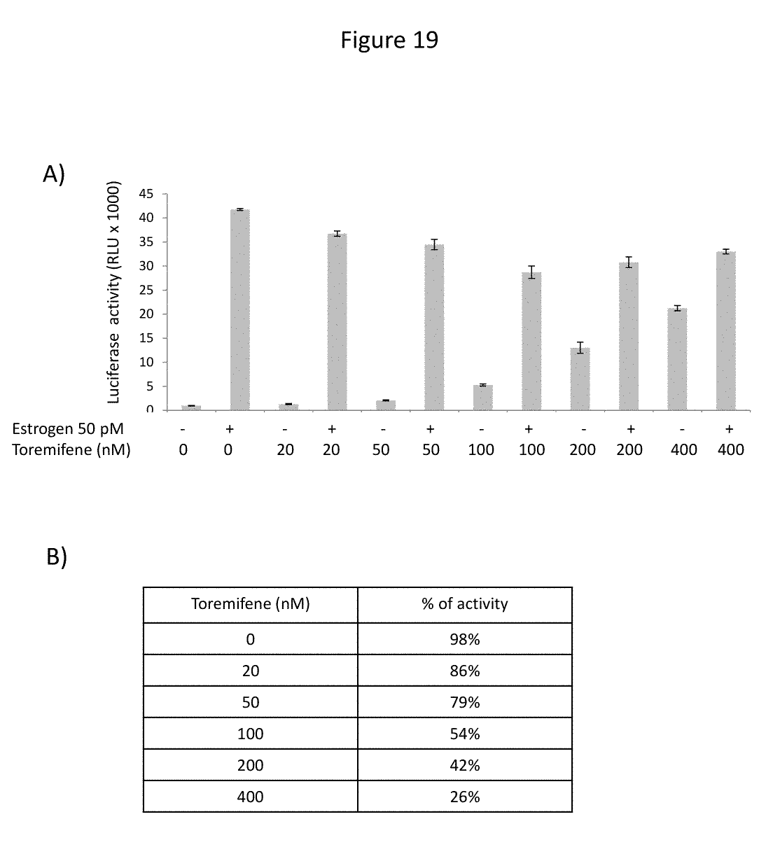

In order to measure the inhibitory activity of toremifene, we transfected an expression plasmid of estrogen receptor into the 3×ERE-luc clone #4 reporter assay cells. The cells were treated with a serial dilution of toremifene in the presence or absence of 50 pM of estrogen. The reporter activity was measured 24 hours post-transfection (FIG. 19A). To calculate the activity of estrogen receptor exposed to estrogen in the presence of tamoxifen, we used the following formula;

Activity of estrogen receptor=[(Luciferase activity of cells treated with estrogen in the presence of toremifene−Luciferase activity of cells treated with toremifene)/(Luciferase activity of cells treated with estrogen−Luciferase activity of cells without treatment)]×100%

In the FIG. 19B, the data clearly show that the activity of estrogen receptor decreased as the concentration of toremifene increased. Therefore, we demonstrated that the activity of estrogen receptor can be calculated from luciferase reporter assay using the above stated formula.

### c) Raloxifene

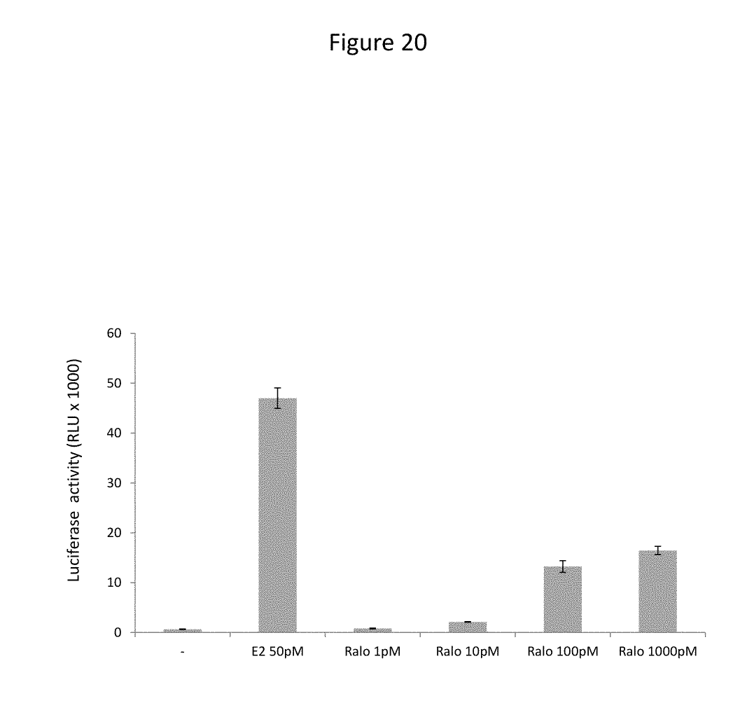

In order to examine the estrogen-like action of raloxifene, we transfected an expression plasmid of estrogen receptor into the 3×ERE-luc clone #4 reporter assay cells. The cells were treated with 50 pM of estrogen or a serial dilution of raloxifene. The reporter activity was measured 24 hours post-transfection (FIG. 20). The cells responded to 50 pM of estrogen and gave a robust induction of luciferase reporter activity. The reporter activity also increased in a dose dependent manner upon raloxifene treatment. This observation demonstrated that the estrogen-like activity of raloxifene existed in our reporter cell lines.

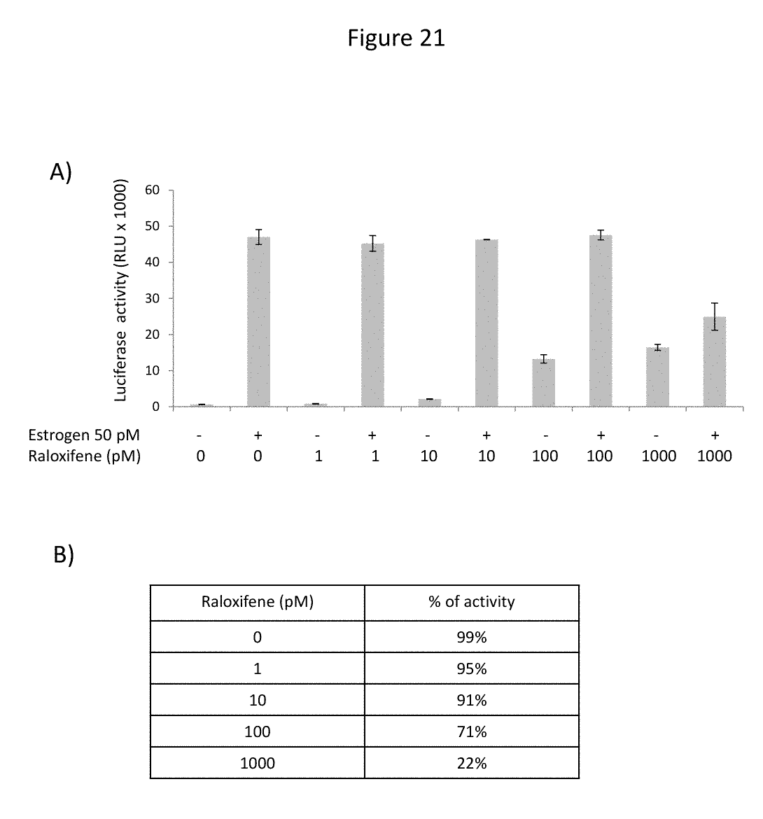

In order to measure the inhibitory activity of raloxifene, we transfected an expression plasmid of estrogen receptor into the 3×ERE-luc clone #4 reporter assay cells. The cells were treated with a serial dilution of raloxifene in the presence or absence of 50 pM of estrogen. The reporter activity was measured 24 hours post-transfection (FIG. 21A). To calculate the activity of estrogen receptor exposed to estrogen in the presence of raloxifene, we used the following formula;

Activity of estrogen receptor=[(Luciferase activity of cells treated with estrogen in the presence of raloxifene−Luciferase activity of cells treated with raloxifene)/(Luciferase activity of cells treated with estrogen−Luciferase activity of cells without treatment)]×100%

In the FIG. 21B, the data clearly show that the activity of estrogen receptor decreased as the concentration of raloxifene increased. Therefore, we demonstrated that the activity of estrogen receptor can be calculated from luciferase reporter assay using the above stated formula.

### Example 8 Miniaturization and Automation of the Assay

Assay miniaturization is important for practical clinical diagnostics. It is not only boosting throughput to keep up with day-day demands but also decreasing costs. To improve reproducibility and accuracy of the assay, we also automated assay workflow by using the Multidrop™384 reagent dispenser (from Thermo Fisher Scientific) to dispense the reporter cells into 384-well plates. Acoustic liquid dispenser (from EDC Biosystems) was used to dispense estrogen and/or estrogen receptor inhibitors into the 384-well plate containing transfected reporter cells. The luciferase reporter activity was measured 24 hours post-transfection by adding the luciferase substrate using the Multidrop™384 reagent dispenser.

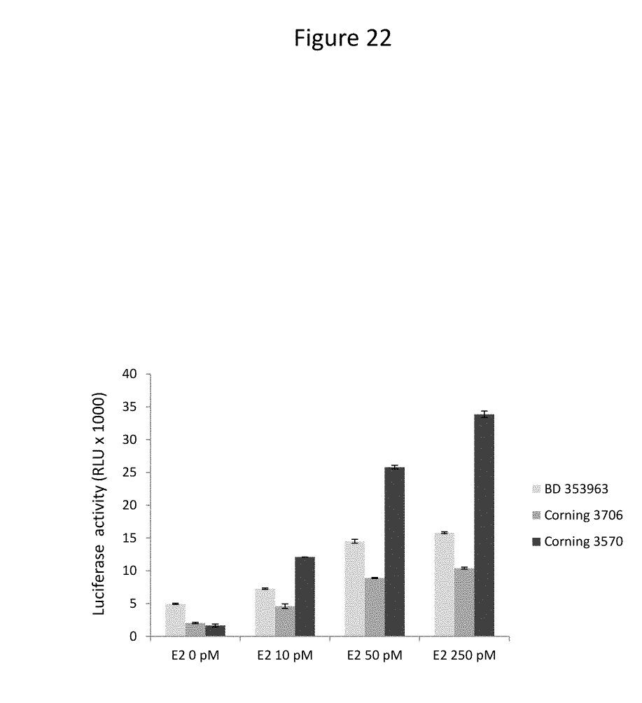

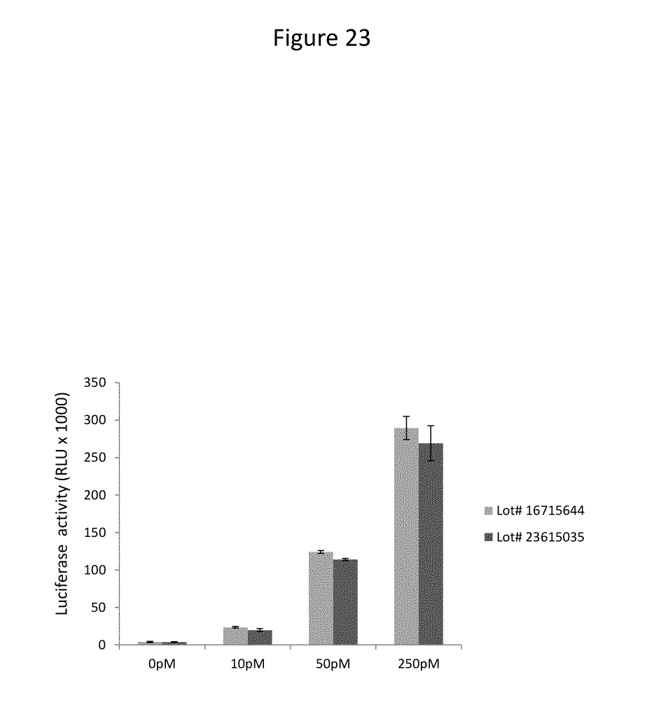

To miniaturize the assay, we tested 3 different types of 384-well plate, including BD #353963, Corning #3706 and Corning #3570. The reporter cells were first mixed with DNA transfection mixture and then dispensed into 3 different types of plates followed by addition of estrogen. The luciferase reporter activity was measured 24 hours post-transfection. In FIG. 22, the data clearly show that Corning #3570 384-well plate gave the best luciferase reporter activity upon exposure among the others. Therefore, the Corning #3570 384-well plate is a preferred embodiment to run the estrogen receptor assay in a clinical laboratory. In addition, we performed the same experiment using this plate from two different lots to examine the lot to lot variation. The same observation was found between them (FIG. 23).

### Example 9 Estrogen Receptor Assay Transfected with Linear Expression Cassette of Wild-Type ESR1 in the 384-Well Format

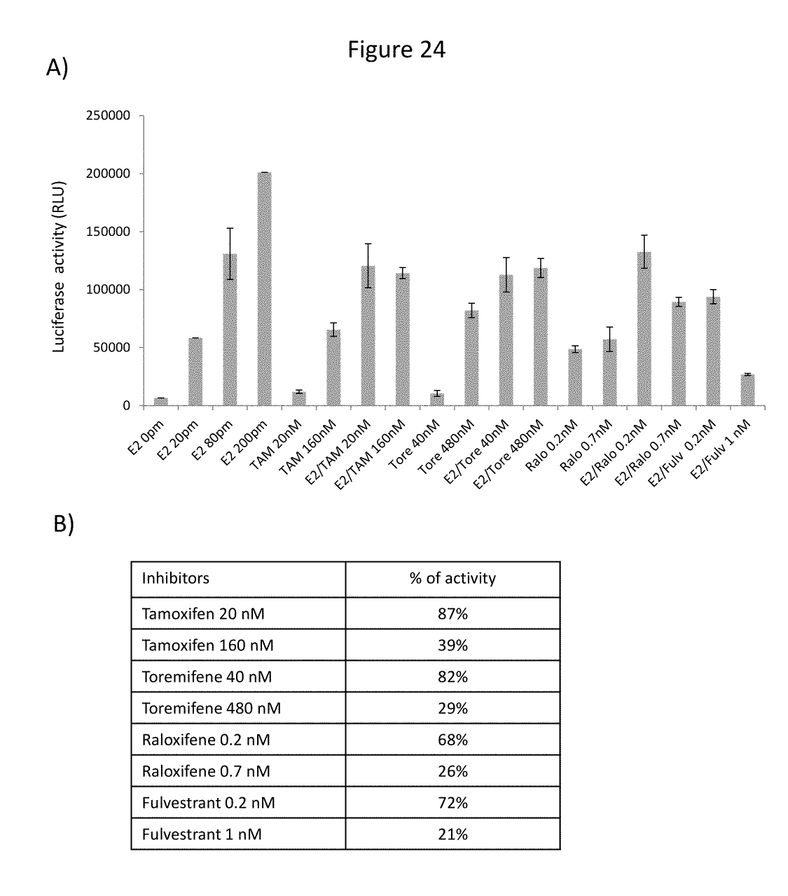

In the previous example, we successfully miniaturized the estrogen receptor assay into the 384-well format. In order to further evaluate the assay, we first transfected the linear expression cassette of wild-type ESR1 DNA into the reporter assay cells followed by treatment with estrogen and its inhibitors using the “all-in-one” method. The luciferase reporter activity was measured 24 hours post-transfection. Two different concentrations of inhibitors were picked and used based on the dose-response curve of inhibitors (Example 3d and 5). The low concentration of inhibitor corresponded to the concentration where treated cells retained more than 50% of estrogen receptor activity upon estrogen exposure. The high concentration of inhibitor corresponded to the concentration where treated cells retained less than 50% of estrogen receptor activity upon estrogen exposure. Since estrogen receptor inhibitors, including tamoxifen, toremifene and raloxifene, were partial agonists, the transfected cells were also treated with inhibitors alone without estrogen exposure. These luciferase reporter activities, which represented their agonistic activity, were used in the formula, as illustrated in Example 5, to calculate the percentage of activity of estrogen receptor upon inhibitor treatment. FIG. 24A showed the luciferase activity of cells transfected with linear ESR1 DNA in different treatment conditions. The reporter assay cells responded to estrogen exposure in a dose dependent manner. FIG. 24B showed the calculated percentage of activity of estrogen receptor after treatment of inhibitors. This result clearly showed that our 3×ERE-luc clone #4 is capable of measuring the activity of transfected estrogen receptor and its response toward the inhibitors.

### Example 10 Case Studies

Mutations in the ER gene (ESR1) have been described in advanced breast cancers that had been exposed to previous therapy with aromatase inhibitors (AIs), drugs that suppress estrogen in postmenopausal women through inhibition of androgen aromatization. ESR1 mutations are also detectable in primary breast cancer and are found at higher frequency after the development of hormone resistance. Most of the ESR1 mutations occur in a hotspot region within the ligand-binding domain (“LBD”) of ER. Functional studies of these LBD ESR1 mutations demonstrated that they constitutively activate the ER in a ligand-independent fashion. Hence, cancers with these ESR1 mutations would be predicted to be resistant to AIs because these therapies work by depriving ligand. However, little is known about the effect of LBD ESR1 mutations on sensitivity to estrogen receptor inhibitors such as selective ER modulators including tamoxifen, toremifene and raloxifene, and a selective ER down-regulator, fulvestrant. In addition, more than 100 mutations affecting other region of ER were reported and published in COSMIC database (Catalogue of Somatic Mutations in Cancer). No functional role of these mutations on ER activity or ER sensitivity in response to inhibitors is known. Therefore, we used our validated estrogen receptor assay to test out some of these mutations.

a) 344insC of ESR1

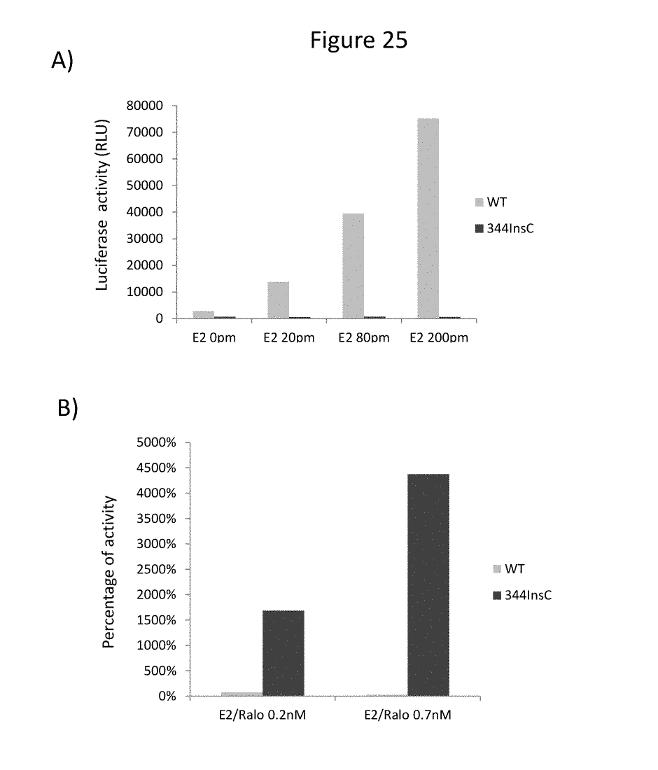

344insC corresponds to a mutation of inframe insertion of a codon, GCT, after amino acid of 344. No functional role of this mutation was reported. In order to functionally characterize this mutation, PCR-mediated overlapping extension was employed to construct linear expression cassette of ESR1 carrying this insertion (Example 1B). We first transfected the linear expression cassette of either wild-type ESR1 or mutant ESR1 DNA into the reporter cells followed by treatment with estrogen and its inhibitors using “all-in-one” method. The luciferase reporter activity was measured 24 hours post-transfection. In FIG. 25A, no luciferase activity was observed in the reporter cells transfected with this mutant form of ESR1 upon treatment of estrogen. The 344insC mutant of ESR1 did not respond to estrogen exposure. This result indicated that the estrogen receptor carrying 344insC mutation was transcriptional inactive.

Same observation was found when the cells were treated with the inhibitors including tamoxifen, toremifene and fulvestrant. No partial agonistic activity of these inhibitors was detected (data not shown). However, when these cells were treated with raloxifene alone or raloxifene and estrogen, strong reporter activity was detected (FIG. 25B). This functional study strongly suggested that raloxifene is estrogenic. Clinically, treatment of raloxifene in this patient would cause clonal expansion of cells carrying this mutation because a selective pressure that favors the proliferation of these cells. These cells eventually dominate the tumor and result in cancer progression and therapy resistance. Therefore, treating patient with raloxifene should be avoided. Other treatment options, including AIs, tamoxifen, toremifene and fulvestrant, should be recommended.

### b) V534E of ESR1

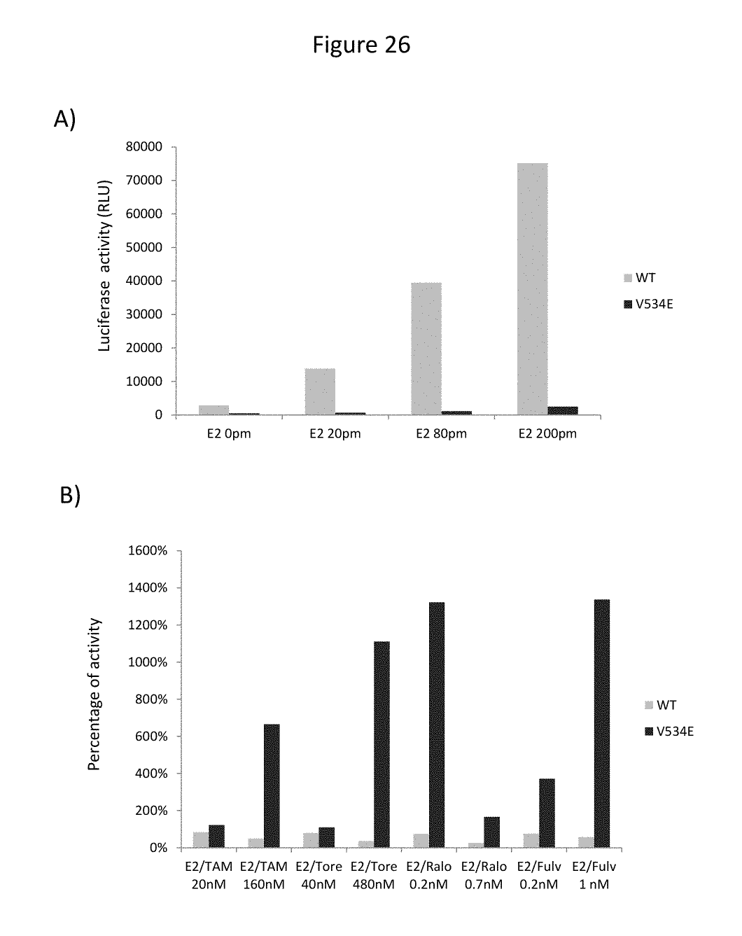

V534E corresponds to a missense mutation of ESR, which changed valine (V) into glutamic acid (E) at amino acid position of 534. No functional role of this mutation was reported. In order to functionally characterize this mutation, PCR-mediated overlapping extension was employed to construct linear expression cassette of ESR1 carrying this insertion (Example 1B). We first transfected the linear expression cassette of either wild-type ESR1 or mutant ESR1 DNA into the reporter cells followed by treatment with estrogen and its inhibitors using “all-in-one” method. The luciferase reporter activity was measured 24 hours post-transfection. In FIG. 26A, the cells transfected with V534E mutant showed very weak induction of luciferase activity upon estrogen exposure. This mutant was not constitutive active. This result indicated that the V534E mutant of estrogen receptor was transcriptional less active compared to wild-type of estrogen receptor.

When these cells were treated with estrogen inhibitors alone or estrogen inhibitors and estrogen, strong reporter activity was detected (FIG. 26B). This functional study strongly suggested that all estrogen inhibitors are estrogenic. Clinically, treatment of estrogen inhibitors in this patient would cause clonal expansion of cells carrying this mutation because a selective pressure that favors the proliferation of these cells. These cells eventually dominate the tumor and result in cancer progression and therapy resistance. Therefore, treating patient with these estrogen inhibitors should be avoided. However, the V534E mutant of estrogen receptor is not constitutive active and its transcriptional activity depends on estrogen. Therefore, AIs treatment should be recommended.

### c) E380Q of ESR1

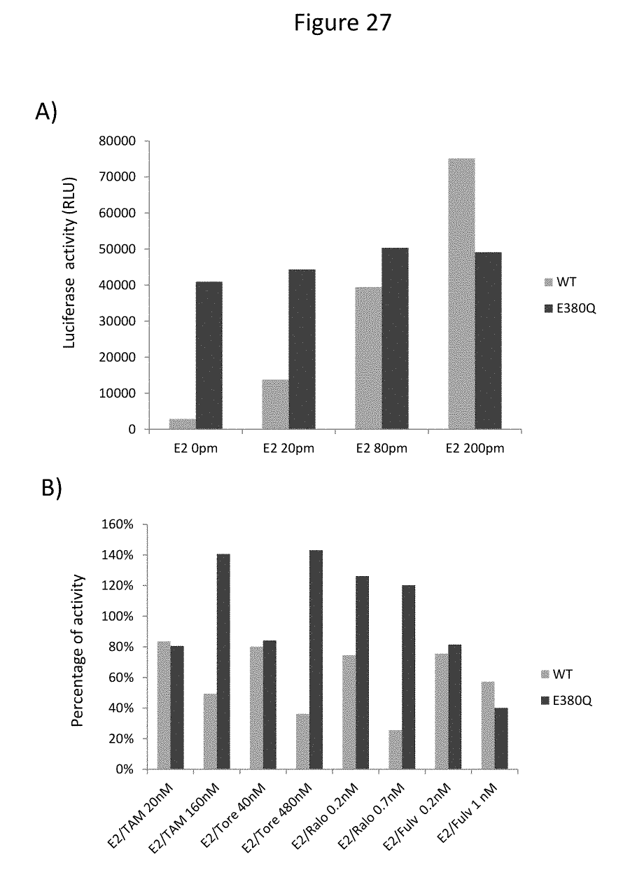

E380Q corresponds to a missense mutation of ESR, which changed glutamic acid (E) into glutamine (Q) at amino acid position of 380. No functional role of this mutation was reported. In order to functionally characterize this mutation, PCR-mediated overlapping extension was employed to construct linear expression cassette of ESR1 carrying this insertion (Example 1B). We first transfected the linear expression cassette of either wild-type ESR1 or mutant ESR1 DNA into the reporter cells followed by treatment with estrogen and its inhibitors using “all-in-one” method. The luciferase reporter activity was measured 24 hours post-transfection. In FIG. 27A, the cells transfected with E380Q mutant showed strong luciferase activity independent to estrogen exposure. This mutant was constitutive active. The result indicated that the E380Q mutant of estrogen receptor was transcriptional active in an estrogen independent manner.

When these cells were treated with the selective ER modulators including tamoxifen, toremifene and raloxifene alone or these modulators and estrogen, no inhibitory activity was detected (FIG. 27B). Instead, weak activation of luciferase activity was detected. This functional study strongly suggested that all the selective ER modulators including tamoxifen, toremifene and raloxifene are slightly estrogenic. However, when these cells were treated with the selective ER down-regulator, fulvestrant, inhibitory activity was detected. Such activity was comparable to the treated cells transfected with wild-type ESR1. This functional study indicated that this mutation did not alter the estrogen receptor to respond to fulvestrant. Clinically, treatment with the selective ER modulators in this patient would cause clonal expansion of cells carrying this mutation because a selective pressure that favors the proliferation of these cells. These cells eventually dominate the tumor and result in cancer progression and therapy resistance. In addition, the E380Q mutant of estrogen receptor is constitutive active and its transcriptional activity is independent to estrogen. Therefore, treating patient with AIs and the selective ER modulators should be avoided. However, the E380Q mutant of estrogen receptor responded normally to fulvestrant. Therefore, fulvestrant treatment should be recommended.

### d) L536P of ESR1

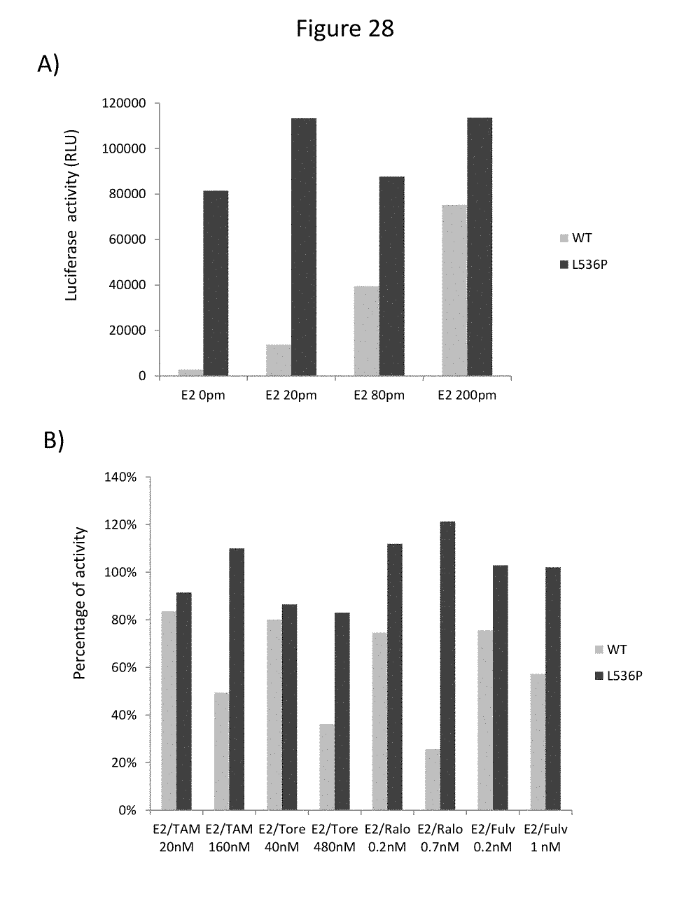

L536P corresponds to a missense mutation of ESR, which changed leucine (L) into proline (P) at amino acid position of 536. No functional role of this mutation was reported. In order to functionally characterize this mutation, PCR-mediated overlapping extension was employed to construct linear expression cassette of ESR1 carrying this insertion (Example 1B). We first transfected the linear expression cassette of either wild-type ESR1 or mutant ESR1 DNA into the reporter cells followed by treatment with estrogen and its inhibitors using “all-in-one” method. The luciferase reporter activity was measured 24 hours post-transfection. In FIG. 28A, the cells transfected with L536P mutant showed strong luciferase activity independent to estrogen exposure. This mutant was constitutive active. The result indicated that the L536P mutant of estrogen receptor was transcriptional active in an estrogen independent manner.

When these cells were treated with all the ER inhibitors, no reduction of luciferase activity was detected (FIG. 28B). This functional study strongly suggested that all these ER inhibitors failed to inhibit the L536Pmutant of estrogen receptor. Clinically, treatment with the ER inhibitors in this patient would cause clonal expansion of cells carrying this mutation because a selective pressure that favors the proliferation of these cells. These cells eventually dominate the tumor and result in cancer progression and therapy resistance. In addition, the L536P mutant of estrogen receptor is constitutive active and its transcriptional activity is independent to estrogen. Therefore, treating patient with AIs and the ER inhibitors should be avoided. Therefore, hormonal treatment by itself is not recommended to treat this patient. Target therapies should be recommended in conjunction with the hormonal treatment.

### Example 11 Construction of Linear Expression Cassette of Human Aromatase (CYP19A1)

Aromatase is a cytochrome P450 (CYP19A1) enzyme that catalyzes the conversion of androgen and testosterone to the aromatic estrogenic steroids estrone and estradiol, respectively. As such, suppression of estrogen biosynthesis by a mechanism of aromatase inhibition represents an effective approach for the treatment of hormone-sensitive breast cancer. The importance of this enzyme in breast cancer development has led to intensive research aiming for inhibitors with the ability to inhibit aromatase, ranging from androstenedione analogues to nonsteroidal derivatives.

In order to study the effect of unknown mutations in human CYP19A1 gene, we generated a linear expression cassette, which contained CMV promoter controlling expression of ESR1 coding sequence followed by terminator and polyadenylation signal. To do so, overlapping extension PCR was employed to construct the linear expression cassette using expression plasmid of human CYP19A1 as PCR template. Using this method, the construction of linear expression cassette takes around 4-8 hours. However, the traditional cloning method to generate expression plasmid takes around 2-4 days. Therefore, making a patient gene in linear expression cassette format is significant for a clinical diagnostic test because of its quick turn-around time.

### a) Construction of Expression Plasmid of Human CYP19A1

cDNA plasmid encoding human CYP19A1 gene was purchased (Dharmacon). We amplified the coding sequence of human CYP19A1 gene by the PCR. We inserted NheI and XbaI restriction enzyme sites to forward and reverse primers respectively for purpose of cloning. The PCR products containing the coding sequence of human CYP19A1 were sub-cloned into the pcDNA3.1 (+) using NheI and XbaI restriction enzymes. The nucleotide sequence of human CYP19A1 was verified by the DNA sequencing. This human CYP19A1 expression plasmid was used as PCR template to construct the linear expression cassettes of wild-type CYP19A1 or mutated forms of CYP19A1.

### b) Generation of Linear Expression Cassette of Human Wild-Type and Mutated CYP19A1

Linear expression cassette of human wild-type CYP19A1 was generated by UF-CMV forward and BGH-UR reverse primers. The amplified products were gel-purified. The DNA concentration was quantitated by the optical density at 260 nm using Nanodrop.

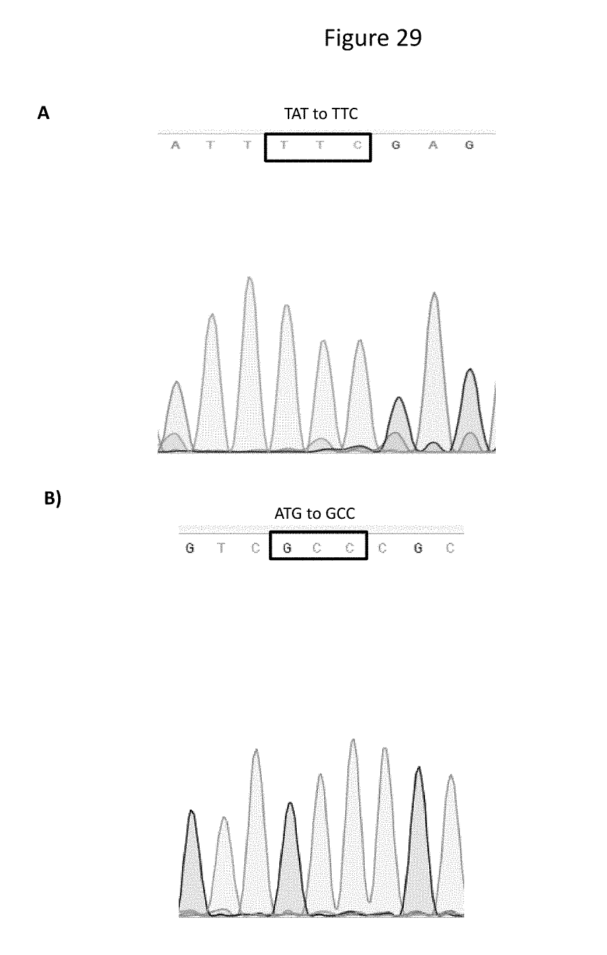

A linear expression cassette of mutated CYP19A1 was generated by PCR mediated overlapping extension method. A pair of forward and reverse primers containing the targeted mutations was designed. The mutated codon (3 nucleotides) was located in the middle of primer flanked by 18 nucleotides on each side. Two separate PCR, named as PCR#1 and PCR#2 in FIG. 1, were performed using UF-CMV forward and mutated reverse primers, or mutated forward and BGH-UR primers. The PCR products were cleaned up by ExoSAP-IT® (Affymetrix) to remove unconsumed dNTPs and primers. These two treated PCR products were mixed together followed by dilution with water. We performed second round of PCR using the diluted PCR mixture, and UF and UR primers (FIG. 1). The amplified products were gel-purified. The DNA concentration was quantitated by the optical density at 260 nm using Nanodrop. The targeted mutations were successfully incorporated in the CYP19A1 gene during the PCR mediated overlapping extension method. The introduced mutations were confirmed by the DNA sequencing (FIG. 29). For Y361F mutant, codon “TAT” encoding tyrosine at amino acid #361 was changed into codon “TTC” encoding phenylalanine (FIG. 29A). Same observation was found when we sequenced the M374A mutant (FIG. 29B). Codon “ATG” encoding methionine at amino acid #374 was successfully changed into codon “GCC” encoding alanine.

### Example 12 Identification of Reporter System to Measure CYP19A1 Activity

Human aromatase (CYP19A1) catalyzes the synthesis of estrogen from androgen with high substrate specificity. Estrogen receptors (ERs) are receptors that are activated by hormone estrogen. It also belongs to DNA-binding transcription factor. In resting cells, the estrogen receptors reside in the cytoplasm. Upon activation by estrogen, the estrogen receptor translocates into the nucleus and binds to DNA to activate transcription of its target genes. ER binds to specific DNA sequences called estrogen response elements (EREs) with high affinity. The consensus ERE DNA sequence has been reported and delineated.

The 3×ERE-luc reporter system has been validated in Example 2C. This reporter system can be used to measure the estrogen receptor activity exposed to estrogen. Since aromatase catalyzes the synthesis of estrogen from testosterone, this 3×ERE-luc reporter construct is capable of measuring aromatase activity. In order to use this reporter system in the clinical diagnostic test, we decided to use the 3×ERE-luc clone #4, which 3×ERE-luc plasmid was stably integrated in the genome of HEK 293 cells.

To validate this cell line, we transfected the cells with expression construct of estrogen receptor or expression constructs of estrogen receptor and aromatase followed by treatment of testosterone. In addition, chemical inhibitors and mutations in aromatase gene with known functional outcome were used in the validation experiments.

### a) Overexpression of Estrogen Receptor or Estrogen Receptor and Aromatase

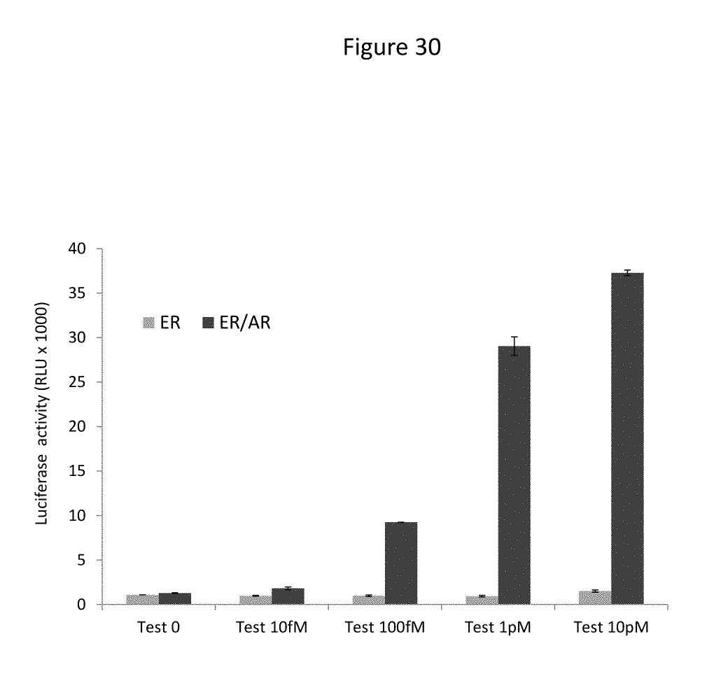

We transfected the clone #4 cells with either expression construct of estrogen receptor or expression constructs of estrogen receptor and aromatase. A serial dilution of testosterone was added to the cells for 24 hours. The luciferase reporter activity was measured. The cells transfected with estrogen receptor alone showed no activation of luciferase activity upon testosterone exposure (FIG. 30). This observation indicated that this clone #4 reporter cells showed no detectable activity of endogenous aromatase. Intriguingly, when we transfected with expression constructs of estrogen receptor and aromatase, a dose dependent activation of luciferase activity was observed upon treatment of testosterone (FIG. 30). Therefore, we concluded that this reporter cells is capable to measure the activity of aromatase.

### b) Validation of Clone #4 by Inhibitors of Aromatase

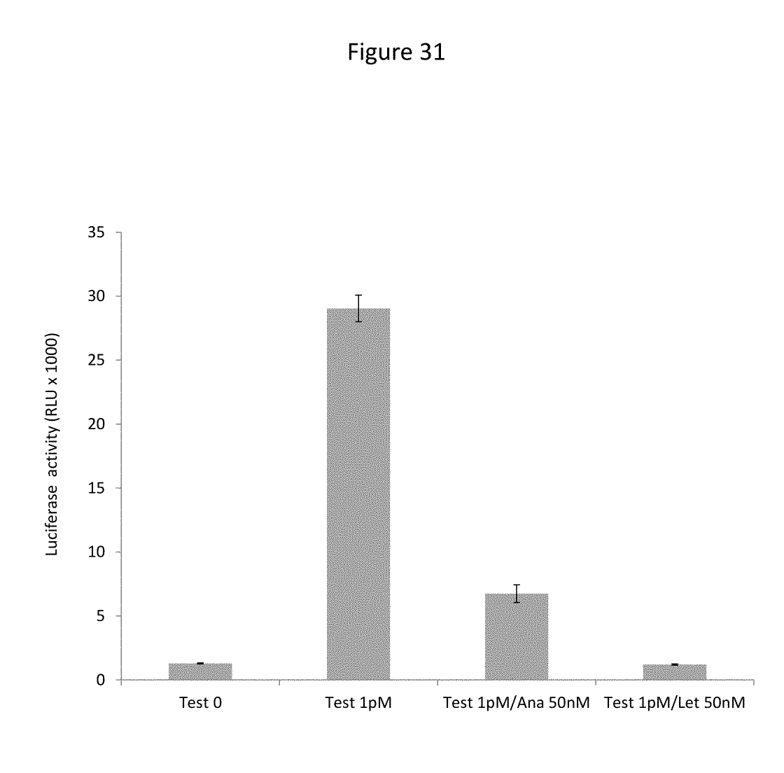

To further validate the reporter cell line, which is potentially used in measuring the activity of aromatase, we transfected the cells with expression constructs of estrogen receptor and aromatase. The transfected cells were treated with testosterone in the presence or absence of aromatase inhibitors, anastrozole and letrozole, for 24 hours (FIG. 31). In the absence of aromatase inhibitors, we detected high luciferase reporter activity upon testosterone exposure. Such increase of reporter activity was reduced by treating the transfected cells with inhibitors, such as anastrozole and letrozole. This observation indicated that this reporter cell lines can be used to measure the response of aromatase toward inhibitors.

### c) Validation of Clone #4 by Mutations, R19211 and R435C, of Aromatase

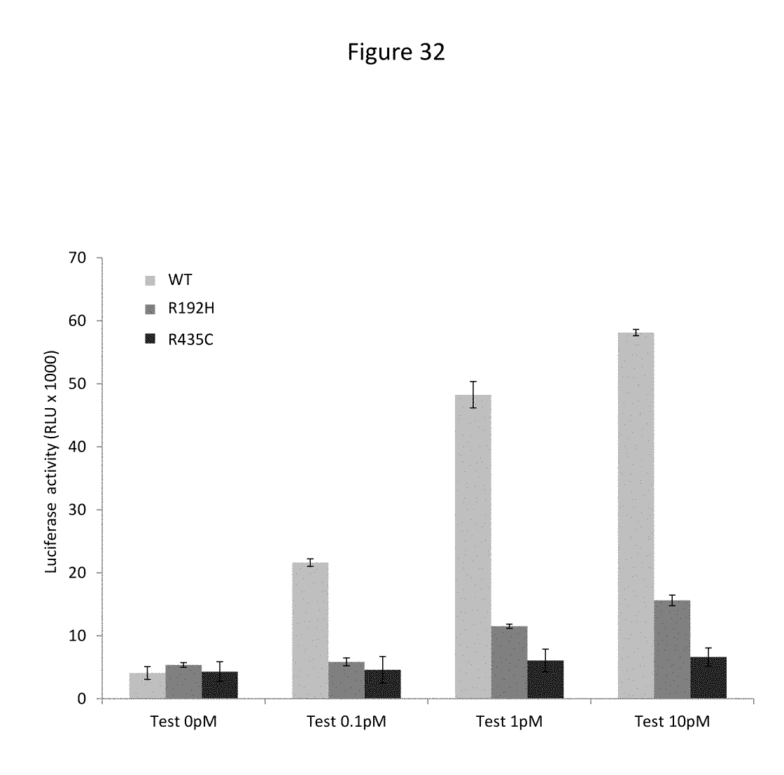

Missense mutations, R192H and R435C, of aromatase have been shown to greatly reduce its enzymatic activity. In addition to validate this reporter cell lines by overexpressing estrogen receptor/aromatase and using inhibitors of aromatase, we further challenged this reporter cells using mutants of aromatase. We transfected the cells with expression constructs of estrogen receptor and aromatase. The transfected cells were treated with a serial dilution of testosterone. The luciferase reporter activity was measured 24 hours post-transfection. The transfected cells expressing R192H mutant showed reduced luciferase reporter activity compared to cells expressing wild-type ER (FIG. 32). Interestingly, the transfected cells expressing R435C mutant showed no induction of luciferase reporter activity upon testosterone exposure. This observation indicated that this reporter cell can be used to measure the effect of mutation on aromatase activity.

### Example 13 Generation of Reporter Cells Stably Expressing Estrogen Receptor

### a) Selection of Stable Cell Clones Expressing Estrogen Receptor

In Example 12, we validated the 3×ERE-luc clone #4 cells to measure the activity of aromatase. However, these cells showed no detectable level of endogenous estrogen receptor. In order to simplify the cell-based assay for aromatase, we decided to generate a single cell clone which is stably expressing estrogen receptor. To do so, we transfected expression construct of estrogen receptor into the 3×ERE-luc clone #4 cells. Antibiotic, G418, was added to the culture medium 48 hours post-transfection. Any cells survived through the selection process suggested that expression construct of estrogen receptor was integrated into the genome.

b) Characterization of Single Cell Clones after G418 Selection

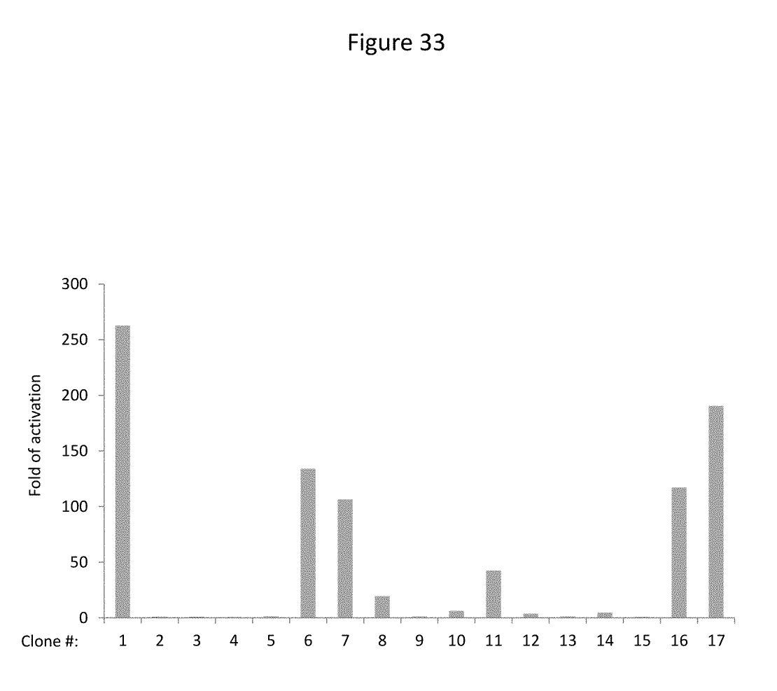

Seventeen single cell clones were picked from the selection plate and were expanded. In order to confirm the expression of estrogen receptor, these single cell clones were exposed to estrogen for 24 hours. The luciferase reporter activity was measured by Nano-Glo® Luciferase assay (Promega) (FIG. 33). Ten out of Seventeen clones showed detectable luciferase reporter activity upon addition of estrogen. Among these 10 clones, clone #1 showed the best fold of induction. Therefore, this clone was used for further analysis.

### c) Clone #1 to Measure Activity of Aromatase

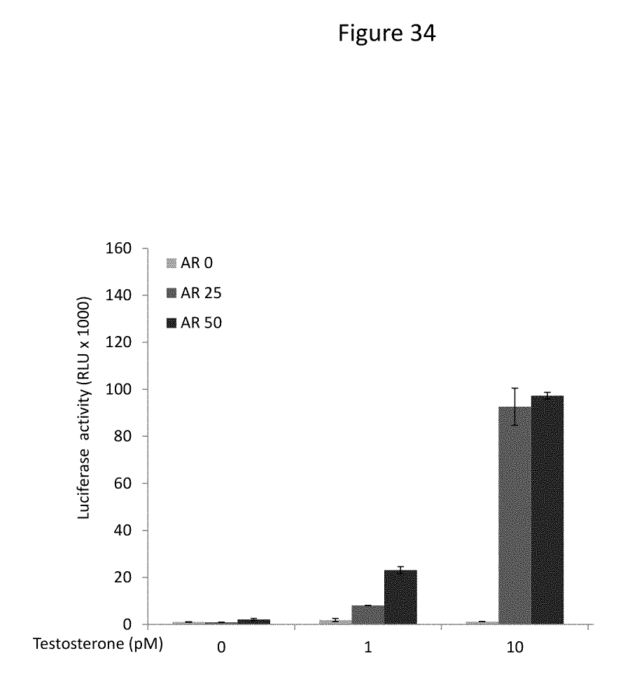

Clone #1 cells were transfected with linear expression cassette of aromatase. After 24 hours, testosterone was added to the cells for 6 hours. The luciferase reporter activity was measured (FIG. 34). No luciferase activity was detected when clone #1 cells were not transfected with aromatase DNA. The cells, which were transfected with either 25 ng or 50 ng of aromatase linear DNA, showed robust stimulation of reporter upon addition of testosterone. This observation indicated that there is no detectable endogenous activity of aromatase. The detected aromatase activity is due to the transient transfected aromatase. This clone responded to testosterone in a dose-dependent manner, which suggested that it can be used to quantitatively measure the activity of aromatase.

### Example 14 Optimization of Cell-Based Assay for Aromatase Using Clone #1

In example 13, we successfully generated stable cell clones containing 3×ERE-luc reporter plasmid and expression plasmid of estrogen receptor. Among them, clone #1 showed the best response toward testosterone exposure. In order to use this clone #1 as a clinical assay, we performed optimization such as duration of treatment and treatment methodology.

### a) Exposure Duration—4, 6 and 24 Hours

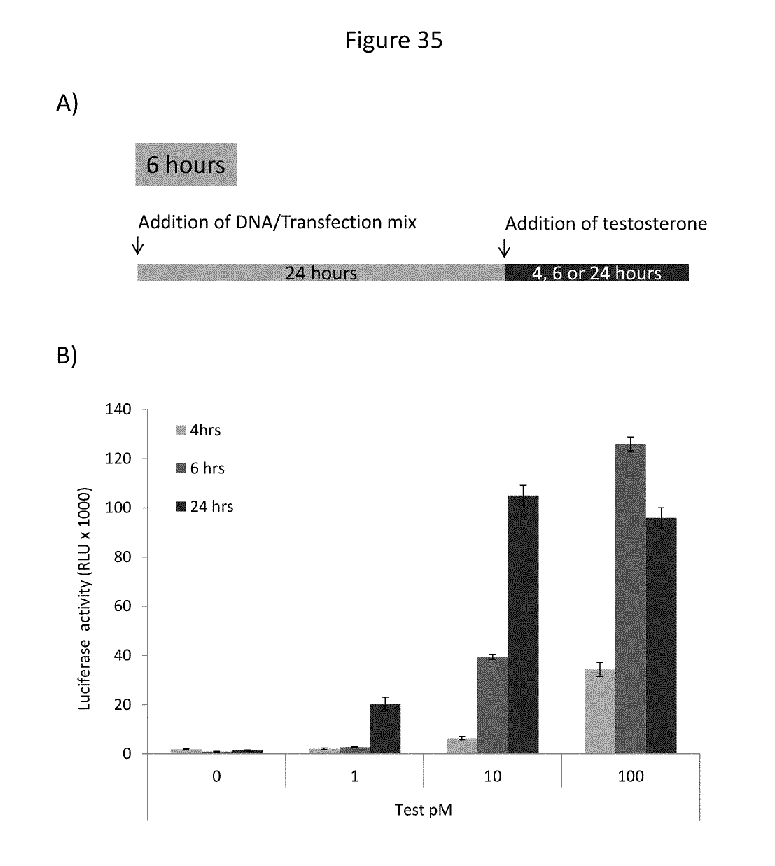

The clone #1 cells were first transfected with 50 ng of linear expression cassette of aromatase. Testosterone was added to the cells 24 hours post-transfection (FIG. 35A). Reporter assay was performed 4, 6 and 24 hours after the exposure. All of them showed a dose-dependent response toward testosterone exposure (FIG. 35B). In this experiment, a better response was found when the cells were treated with testosterone for 24 hours. A robust reporter signal was detected even at 1 pM of testosterone.

### b) Different Exposure Method

To further explore another possibility to reduce the assay turnaround time, we explored different exposure methods. The first one showed in the Example 14a was addition of testosterone to the cells 24 hours post-transfection for 4, 6 and 24 hours. The other method was treating the cells with testosterone when we added the DNA transfection complex to the cells. The reporter activity was measured 24 hours post-transfection (FIG. 35A). The second method was named as “all-in-one”. FIG. 35B showed that all-in-one method gave a good dose response toward testosterone exposure. Therefore, we decided to use the all-in-one method in our aromatase assay.

### Example 15 Dose Dependent Response of Aromatase Inhibitors, Anastrozole and Letrozole

### a) Anastrozole

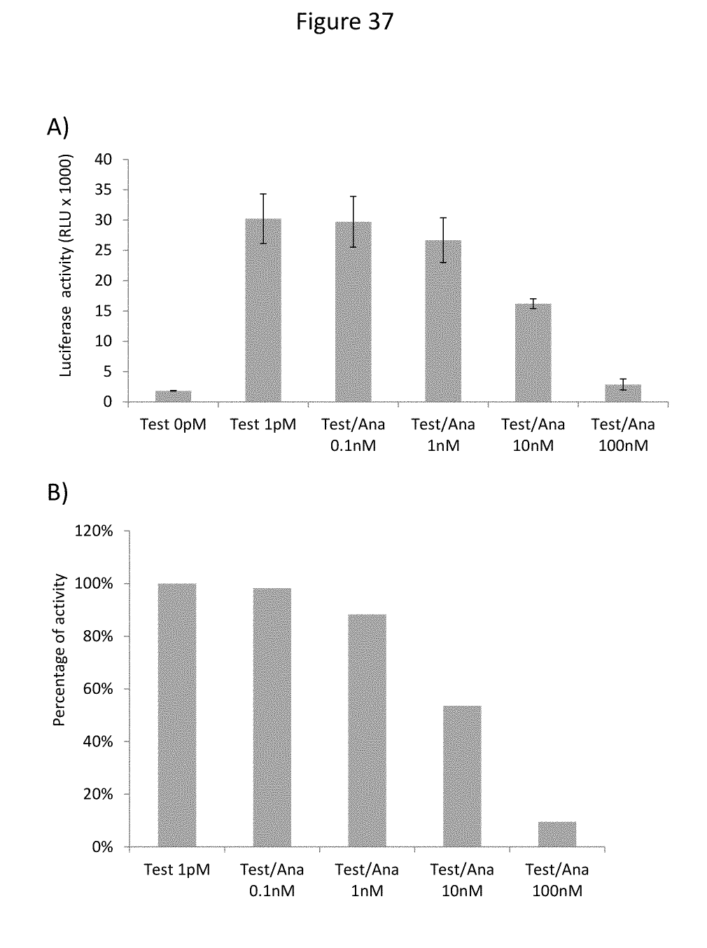

In order to measure the inhibitory activity of anastrozole, we transfected linear expression cassette of aromatase into the clone #1 reporter cells. The cells were treated with either 1 pM of testosterone or 1 pM of testosterone with a serial dilution of anastrozole. The reporter activity was measured 24 hours post-transfection (FIG. 37A). To calculate the activity of aromatase exposed to testosterone in the presence of anastrozole, we used the following formula;

Activity of aromatase=[Luciferase activity of cells treated with testosterone in the presence of anastrozle/Luciferase activity of cells treated with testosterone]×100%

In the FIG. 37B, it clearly showed that the activity of aromatase decreased as the concentration of anastrozole increased. Therefore, we concluded that the activity of aromatase can be calculated from luciferase reporter assay using the above stated formula. The clone #1 cells are capable to measure the response of aromatase toward anastrozole.

### b) Letrozole

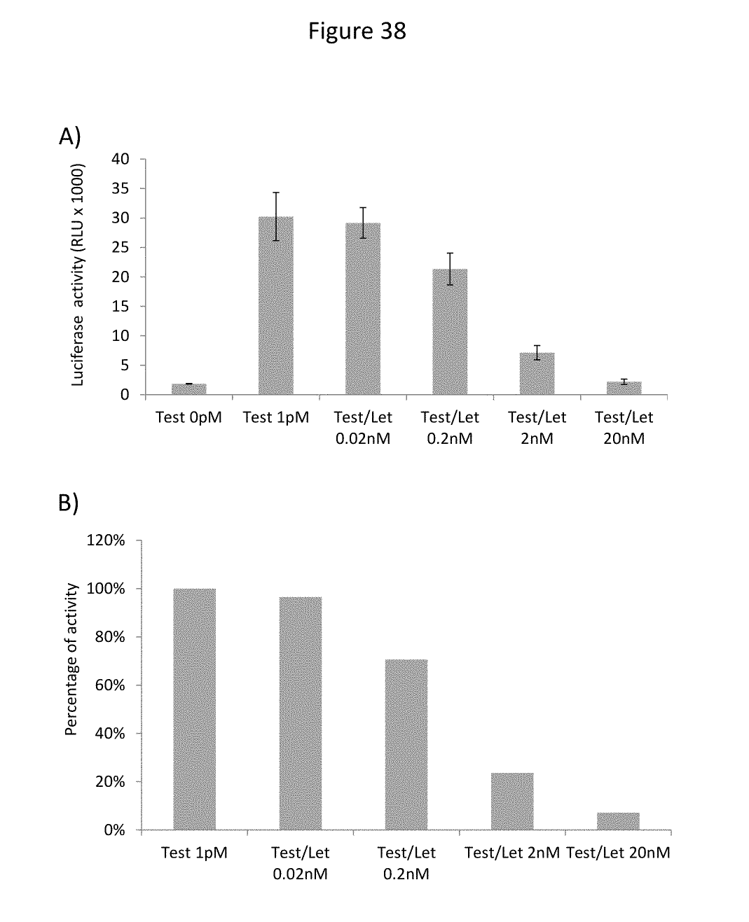

In order to measure the inhibitory activity of letrozole, we transfected linear expression cassette of aromatase into the clone #1 reporter cells. The cells were treated with either 1 pM of testosterone or 1 pM of testosterone with a serial dilution of letrozole. The reporter activity was measured 24 hours post-transfection (FIG. 38A). To calculate the activity of aromatase exposed to

testosterone in the presence of letrozole, we used the following formula;

Activity of aromatase=[Luciferase activity of cells treated with testosterone in the presence of letrozole/Luciferase activity of cells treated with testosterone]×100%

In the FIG. 38B, it clearly showed that the activity of aromatase decreased as the concentration of letrozole increased. Therefore, we concluded that the activity of aromatase can be calculated from luciferase reporter assay using the above stated formula. The clone #1 cells are capable to measure the response of aromatase toward letrozole.

### Example 16 Aromatase Assay Transfected with Linear Expression Cassette of Wild-Type CYP19A1 in the 384-Well Format

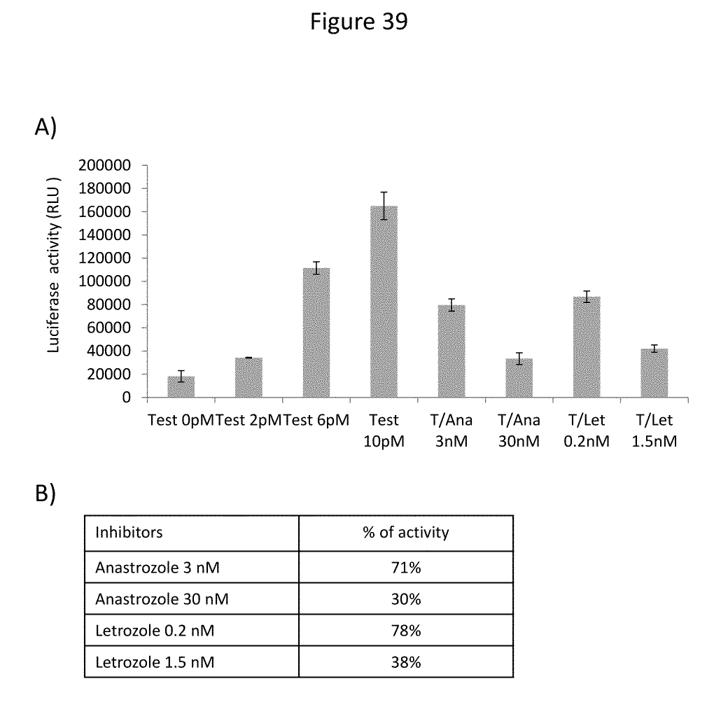

In example 6, we successfully miniaturized the estrogen receptor assay into the 384-well format. In order to further evaluate the aromatase assay, we first transfected the linear expression cassette of wild-type CYP19A1 DNA into the reporter cells (Clone #1) followed by treatment with testosterone and its inhibitors using “all-in-one” method. The luciferase reporter activity was measured 24 hours post-transfection. Two different concentrations of inhibitors were picked and used based on the dose-response curve of inhibitors (Example 15). The low concentration of inhibitor corresponded to the concentration where treated cells retained more than 50% of aromatase activity upon testosterone exposure. The high concentration of inhibitor corresponded to the concentration where treated cells retained less than 50% of aromatase activity upon testosterone exposure. FIG. 39A showed the luciferase activity of cells transfected with linear CYP19A1 DNA in different treatment conditions. The reporter cells responded to testosterone exposure in a dose dependent manner. FIG. 39B showed the calculated percentage of activity of aromatase after treatment of inhibitors. This result clearly showed that clone #1 cells are capable of measuring the activity of transfected aromatase and its response toward the inhibitors.

### Example 17 Case Studies

### a) R192H of CYP19A1

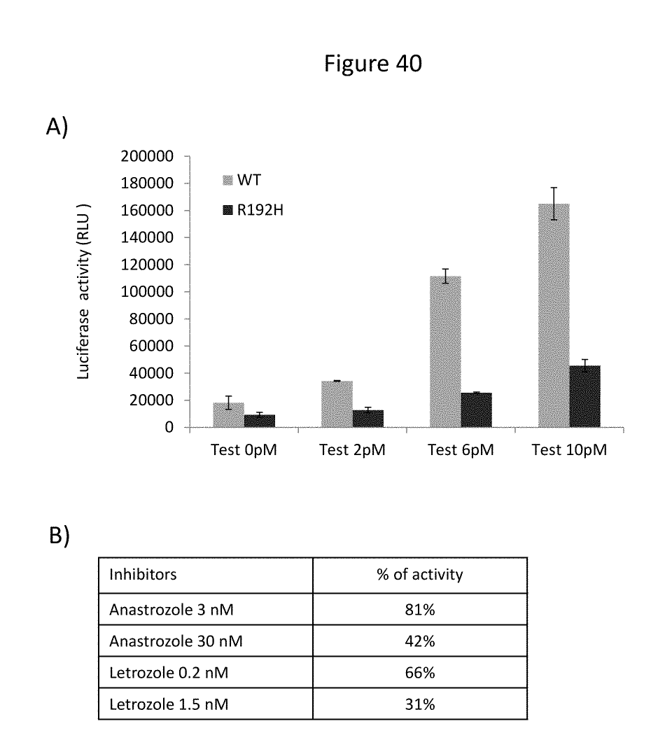

R192H corresponds to a missense mutation of CYP19A1, which changed arginine (R) into histidine (H) at amino acid position of 192. In order to functionally characterize this mutation, PCR-mediated overlapping extension was employed to construct linear expression cassette of aromatase carrying this mutation. We first transfected the linear expression cassette of either wild-type aromatase or mutant aromatase DNA into the reporter cells followed by treatment with testosterone and its inhibitors using “all-in-one” method. The luciferase reporter activity was measured 24 hours post-transfection. In FIG. 40A, reduced luciferase activity was observed in the reporter cells transfected with this mutant form of aromatase upon treatment of testosterone. This result indicated that the R192H mutation diminished the enzymatic activity of aromatase.

When these cells were treated with aromatase inhibitors in the presence of testosterone, reduction of reporter activity was detected. We used the formula as illustrated in the above-mentioned example to calculate the activity of aromatase. The result was summarized in the FIG. 40B. Although the mutation diminished the enzymatic activity of aromatase, it did not alter the activity of aromatase inhibitors. Clinically, treatment of aromatase inhibitors in this patient should not cause clonal expansion of cells, which carry this mutation. Therefore, AI treatment should be recommended.

### b) R435C of CYP19A1

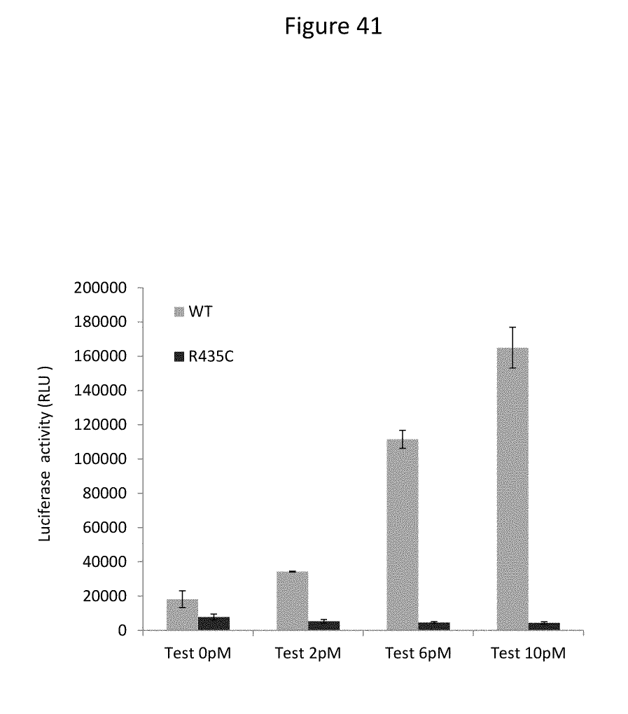

R435C corresponds to a missense mutation of CYP19A1, which changed arginine (R) into cysteine (C) at amino acid position of 435. In order to functionally characterize this mutation, PCR-mediated overlapping extension was employed to construct linear expression cassette of aromatase carrying this mutation. We first transfected the linear expression cassette of either wild-type aromatase or mutant aromatase DNA into the reporter cells followed by treatment with testosterone and its inhibitors using “all-in-one” method. The luciferase reporter activity was measured 24 hours post-transfection. In FIG. 41, no increase in luciferase activity was observed in the reporter assay cells transfected with this mutant form of aromatase upon treatment of testosterone. This result indicated that the R435C mutation completely abolished the enzymatic activity of aromatase.

Since the R435C mutant of aromatase is enzymatically dead, we are not able to measure the inhibitory activity of aromatase inhibitor. Clinically, treatment of aromatase inhibitors in this patient should not cause clonal expansion of cells, which carry this mutation. Therefore, AI treatment should be recommended.

### Materials and Methods

### Construction of Expression Plasmid of ESR1

Human wild-type ESR1 cDNA plasmid was ordered from Open Biosystems (GE Dharmacon). A pair of PCR primers was designed to amplify the coding region as shown below;

hERNheI F:
(SEQ ID NO: 1)
TGGCTAGCCGCCACCATGACCATGACCCTCCACACC
hERXhoI R:
(SEQ ID NO: 2)
GACTCGAGTCAGACCGTGGCAGGGAAACC

Restriction enzyme sites of NheI and XhoI were added to the forward and reverse primers respectively. Coding sequence of wild-type ESR1 was PCR amplified using hERNheI F and hERXhoI R primers from cDNA plasmid using Q5® high-fidelity DNA polymerase (NEB). The amplified PCR product was run on agarose gels and purified using DNA gel purification kit from Qiagen. The gel purified PCR products and pcDNA3.1 DNA vector were treated with NheI and XhoI restriction enzymes at 37° C. for 2 hours. The digested products were run on agarose gels and purified using DNA gel purification kit from Qiagen. The PCR fragment containing coding sequence of wild-type ESR1 was ligated with linearized pcDNA3.1 DNA vector using fast ligation kit from NEB. The ligated products were transformed into Top10 competent cell (Invitrogen). The transformed competent cells were selected using LB plate containing ampicillin for 16 hours at 37° C. The ampicillin resistant clones were cultured in 2 mL of LB medium with ampicillin for 16 hours at 37° C. DNA was extracted from the bacteria culture using DNA mini-preparation kit from Qiagene. The wild-type ESR1 expression plasmid was confirmed by both restriction enzyme digestion and DNA sequencing.

### Construction of Expression Plasmid of Aromatase (CYP19A1)

Human wild-type CYP19A1 cDNA plasmid was ordered from Open Biosystems (GE Dharmacon). A pair of PCR primers was designed to amplify the coding region as shown below;

hCYP19A1 NheI F:
(SEQ ID NO: 3)
TGGCTAGCCGCCACCATGGTTTTGGAAATGCTGAAC
hCYP19A1 XbaI R:
(SEQ ID NO: 4)
CCTCTAGACTAGTGTTCCAGACACCTGTC

Restriction enzyme sites of NheI and XbaI were added to the forward and reverse primers respectively. Coding sequence of wild-type CYP19A1 was PCR amplified using hCYP19A1 NheI F and hCYP19A1 XbaI R primers from cDNA plasmid using Q5® high-fidelity DNA polymerase (NEB). The amplified PCR product was run on agarose gels and purified using DNA gel purification kit from Qiagen. The gel purified PCR products and pcDNA3.1 DNA vector were treated with NheI and XbaI restriction enzymes at 37° C. for 2 hours. The digested products were run on agarose gels and purified using DNA gel purification kit from Qiagen. The PCR fragment containing coding sequence of wild-type CYP19A1 was ligated with linearized pcDNA3.1 DNA vector using fast ligation kit from NEB. The ligated products were transformed into Top10 competent cell (Invitrogen). The transformed competent cells were selected using LB plate containing ampicillin for 16 hours at 37° C. The ampicillin resistant clones were cultured in 2 mL of LB medium with ampicillin for 16 hours at 37° C. DNA was extracted from the bacteria culture using DNA mini-preparation kit from Qiagene. The wild-type ESR1 expression plasmid was confirmed by both restriction enzyme digestion and DNA sequencing.

### Construction of Linear Expression Cassette of Wild-Type ESR1 and its Mutants

The following primers were used to construct the linear expression cassette of wild-type ESR1 and its mutants;

(SEQ ID NO: 5)
UF-CMV F: GCGTTCGCTAAGCGTAGCTAGCGATGTACGGGCCAGATA
(SEQ ID NO: 6)
UF: GCGTTCGCTAAGCGTAGCTAG
(SEQ ID NO: 7)
UR-BGH R: TCTGATACGTCTCGACGCACTCTCCCAGCATGCCTGCTATTG
(SEQ ID NO: 8)
UR: TCTGATACGTCTCGACGCACTC
(SEQ ID NO: 9)
ER K303R F: ATGATCAAACGCTCTAAGAGGAACAGCCTGGCCTTGTCC
(SEQ ID NO: 10)
ER K303R R: CAAGGCCAGGCTGTTCCTCTTAGAGCGTTTGATCAT
(SEQ ID NO: 11)
ER Y537S F: AAGAACGTGGTGCCCCTCTCTGACCTGCTGCTGGAGATG
(SEQ ID NO: 12)
ER Y537S R: CATCTCCAGCAGCAGGTCAGAGAGGGGCACCACGTTCTT
(SEQ ID NO: 13)
ER S305E F: AAACGCTCTAAGAAGAACGAGCTGGCCTTGTCCCTGACG
(SEQ ID NO: 14)
ER S305E R: CGTCAGGGACAAGGCCAGCTCGTTCTTCTTAGAGCGTTT
(SEQ ID NO: 15)
ER D351Y F: TTACTGACCAACCTGGCATACAGGGAGCTGGTTCACATG
(SEQ ID NO: 16)
ER D351Y R: CATGTGAACCAGCTCCCTGTATGCCAGGTTGGTCAGTAA
(SEQ ID NO: 17)
ER 344insC F: GAAGCTTCGATGATGGGCGCTTTACTGACCAACCTGGCA
(SEQ ID NO: 18)
ER 344insC R: TGCCAGGTTGGTCAGTAAAGCGCCCATCATCGAAGCTTC
(SEQ ID NO: 19)
ER E380Q F: GATCAGGTCCACCTTCTACAATGTGCCTGGCTAGAGATC
(SEQ ID NO: 20)
ER E380Q R: GATCTCTAGCCAGGCACATTGTAGAAGGTGGACCTGATC
(SEQ ID NO: 21)
ER S463P F: GGAGTGTACACATTTCTGCCCAGCACCCTGAAGTCTCTG
(SEQ ID NO: 22)
ER S463P R: CAGAGACTTCAGGGTGCTGGGCAGAAATGTGTACACTCC
(SEQ ID NO: 23)
ER R503W F: CTGCAGCAGCAGCACCAGTGGCTGGCCCAGCTCCTCCTC
(SEQ ID NO: 24)
ER R503W R: GAGGAGGAGCTGGGCCAGCCACTGGTGCTGCTGCTGCAG
(SEQ ID NO: 25)
ER V534E F: ATGAAGTGCAAGAACGTGGAGCCCCTCTATGACCTGCTG
(SEQ ID NO: 26)
ER V534E R: CAGCAGGTCATAGAGGGGCTCCACGTTCTTGCACTTCAT
(SEQ ID NO: 27)
ER P535H F: AAGTGCAAGAACGTGGTGCACCTCTATGACCTGCTGCTG
(SEQ ID NO: 28)
ER P535H R: CAGCAGCAGGTCATAGAGGTGCACCACGTTCTTGCACTT
(SEQ ID NO: 29)
ER L536P F: TGCAAGAACGTGGTGCCCCCCTATGACCTGCTGCTGGAG
(SEQ ID NO: 30)
ER L536P R: CTCCAGCAGCAGGTCATAGGGGGGCACCACGTTCTTGCA
(SEQ ID NO: 31)
ER L536R F: TGCAAGAACGTGGTGCCCCGCTATGACCTGCTGCTGGAG
(SEQ ID NO: 32)
ER L536R R: CTCCAGCAGCAGGTCATAGCGGGGCACCACGTTCTTGCA
(SEQ ID NO: 33)
ER L536Q F: TGCAAGAACGTGGTGCCCCAGTATGACCTGCTGCTGGAG
(SEQ ID NO: 34)
ER L536Q R: CTCCAGCAGCAGGTCATACTGGGGCACCACGTTCTTGCA
(SEQ ID NO: 35)
ER D538G F: AACGTGGTGCCCCTCTATGGCCTGCTGCTGGAGATGCTG
(SEQ ID NO: 36)
ER D538G R: CAGCATCTCCAGCAGCAGGCCATAGAGGGGCACCACGTT
(SEQ ID NO: 37)
ER Y537C F: AAGAACGTGGTGCCCCTCTGTGACCTGCTGCTGGAGATG
(SEQ ID NO: 38)
ER Y537C R: CATCTCCAGCAGCAGGTCACAGAGGGGCACCACGTTCTT
(SEQ ID NO: 39)
ER Y537N F: AAGAACGTGGTGCCCCTCAATGACCTGCTGCTGGAGATG
(SEQ ID NO: 40)
ER Y537N R: CATCTCCAGCAGCAGGTCATTGAGGGGCACCACGTTCTT
(SEQ ID NO: 41)
ER E247K F: CGGCTCCGCAAATGCTACAAAGTGGGAATGATGAAAGGT
(SEQ ID NO: 42)
ER E247K R: ACCTTTCATCATTCCCACTTTGTAGCATTTGCGGAGCCG
(SEQ ID NO: 43)
ER R158C F: CCAAATTCAGATAATCGATGCCAGGGTGGCAGAGAAAGA
(SEQ ID NO: 44)
ER R158C R: TCTTTCTCTGCCACCCTGGCATCGATTATCTGAATTTGG
(SEQ ID NO: 45)
ER R243C F: TGCCAGGCCTGCCGGCTCTGCAAATGCTACGAAGTGGGA
(SEQ ID NO: 46)
ER R243C R: TCCCACTTCGTAGCATTTGCAGAGCCGGCAGGCCTGGCA
(SEQ ID NO: 47)
ER T563M F: GGGGCATCCGTGGAGGAGATGGACCAAAGCCACTTGGCC
(SEQ ID NO: 48)
ER T563M R: GGCCAAGTGGCTTTGGTCCATCTCCTCCACGGATGCCCC
(SEQ ID NO: 49)
ER V533M F: AGCATGAAGTGCAAGAACATGGTGCCCCTCTATGACCTG
(SEQ ID NO: 50)
ER V533M R: CAGGTCATAGAGGGGCACCATGTTCTTGCACTTCATGCT
(SEQ ID NO: 51)
ER E423D F: TGTGTAGAGGGCATGGTGGATATCTTCGACATGCTGCTG
(SEQ ID NO: 52)
ER E423D R: CAGCAGCATGTCGAAGATATCCACCATGCCCTCTACACA
(SEQ ID NO: 53)
ER R269C F: AGAATGTTGAAACACAAGTGCCAGAGAGATGATGGGGAG
(SEQ ID NO: 54)
ER R269C R: CTCCCCATCATCTCTCTGGCACTTGTGTTTCAACATTCT
(SEQ ID NO: 55)
ER S102N F: GGTTTCCCCCCACTCAACAACGTGTCTCCGAGCCCGCTG
(SEQ ID NO: 56)
ER S102N R: CAGCGGGCTCGGAGACACGTTGTTGAGTGGGGGGAAACC
(SEQ ID NO: 57)
ER R555H F: CTACATGCGCCCACTAGCCATGGAGGGGCATCCGTGGAG
(SEQ ID NO: 58)
ER R555H R: CTCCACGGATGCCCCTCCATGGCTAGTGGGCGCATGTAG
(SEQ ID NO: 59)
ER R434W F: CTGCTGGCTACATCATCTTGGTTCCGCATGATGAATCTG
(SEQ ID NO: 60)
ER R434W R: CAGATTCATCATGCGGAACCAAGATGATGTAGCCAGCAG
(SEQ ID NO: 61)
ER E419D F: AACCAGGGAAAATGTGTAGATGGCATGGTGGAGATCTTC
(SEQ ID NO: 62)
ER E419D R: GAAGATCTCCACCATGCCATCTACACATTTTCCCTGGTT
(SEQ ID NO: 63)
ER T182A F: ATGGAATCTGCCAAGGAGGCTCGCTACTGTGCAGTGTGC
(SEQ ID NO: 64)
ER T182A R: GCACACTGCACAGTAGCGAGCCTCCTTGGCAGATTCCAT
(SEQ ID NO: 65)
ER V534M F: ATGAAGTGCAAGAACGTGATGCCCCTCTATGACCTGCTG
(SEQ ID NO: 66)
ER V534M R: CAGCAGGTCATAGAGGGGCATCACGTTCTTGCACTTCAT
(SEQ ID NO: 67)
ER L536R F: TGCAAGAACGTGGTGCCCCGCTATGACCTGCTGCTGGAG
(SEQ ID NO: 68)
ER L536R R: CTCCAGCAGCAGGTCATAGCGGGGCACCACGTTCTTGCA
(SEQ ID NO: 69)
ER H6Y F: ACCATGACCATGACCCTCTACACCAAAGCATCTGGGATG
(SEQ ID NO: 70)
ER H6Y R: CATCCCAGATGCTTTGGTGTAGAGGGTCATGGTCATGGT
(SEQ ID NO: 71)
ER A166S F: GGTGGCAGAGAAAGATTGTCCAGTACCAATGACAAGGGA
(SEQ ID NO: 72)
ER A166S R: TCCCTTGTCATTGGTACTGGACAATCTTTCTCTGCCACC
(SEQ ID NO: 73)
ER R548H F: GAGATGCTGGACGCCCACCACCTACATGCGCCCACTAGC
(SEQ ID NO: 74)
ER R548H R: GCTAGTGGGCGCATGTAGGTGGTGGGCGTCCAGCATCTC
(SEQ ID NO: 75)
ER V3921 F: ATCCTGATGATTGGTCTCATCTGGCGCTCCATGGAGCAC
(SEQ ID NO: 76)
ER V3921 R: GTGCTCCATGGAGCGCCAGATGAGACCAATCATCAGGAT
(SEQ ID NO: 77)
ER A551V F: GACGCCCACCGCCTACATGTGCCCACTAGCCGTGGAGGG
(SEQ ID NO: 78)
ER A551V R: CCCTCCACGGCTAGTGGGCACATGTAGGCGGTGGGCGTC

The linear expression cassette of wild-type ESR1 was amplified from the wild-type ESR1 expression plasmid using UF-CMV F and UR-BGH R primers. The amplified PCR product was run on agarose gels and purified using DNA gel purification kit from Qiagen.

The linear expression cassettes of mutant forms of ESR1 were prepared by PCR mediated overlapping extension. Two rounds of PCR amplification were performed. The first round of PCR included two independent PCR using UF-CMV F and mutation specific R primers or UR-BGH R and mutation specific F primers to amplify coding region of ESR1 into two fragments. The PCR products were then treated with ExoSAP-IT from Affymetrix to eliminate the unincorporated primers and dNTPs. The mixture was incubated at 37° C. for 15 minutes followed by 80° C. for 15 minutes. Then, 10 ul of PCR products from each reaction were added to 30 ul of water. 5 ul of the diluted products were used as template to performed second round of PCR. In this PCR, UF and UR primers were added to the reaction to construct the linear expression cassette of ESR1 carrying the desired mutation. The amplified PCR product was run on agarose gels and purified using DNA gel purification kit from Qiagen.

### Construction of Linear Expression Cassette of Wild-Type ESR1 and its Mutants

The following primers were used to construct the linear expression cassette of wild-type CYP19A1 and its mutants;

UF-CMV F:
(SEQ ID NO: 79)
GCGTTCGCTAAGCGTAGCTAGCGATGTACGGGCCAGATA
UF:
(SEQ ID NO: 80)
GCGTTCGCTAAGCGTAGCTAG
UR-BGH R:
(SEQ ID NO: 81)
TCTGATACGTCTCGACGCACTCTCCCAGCATGCCTGCTATTG
UR:
(SEQ ID NO: 82)
TCTGATACGTCTCGACGCACTC
CYP19A1 R192H F:
(SEQ ID NO: 83)
GACGTGTTGACCCTTCTGCATCGTGTCATGCTGGACACC
CYP19A1 R192H R:
(SEQ ID NO: 84)
GGTGTCCAGCATGACACGATGCAGAAGGGTCAACACGTC
CYP19A1 R435C F:
(SEQ ID NO: 85)
CCATTTGGCTTTGGGCCCTGTGGCTGTGCAGGAAAGTAC
CYP19A1 R435C R:
(SEQ ID NO: 86)
GTACTTTCCTGCACAGCCACAGGGCCCAAAGCCAAATGG

The linear expression cassette of wild-type CYP19A1 was amplified from the wild-type CYP19A1 expression plasmid using UF-CMV F and UR-BGH R primers. The amplified PCR product was run on agarose gels and purified using DNA gel purification kit from Qiagen.

The linear expression cassettes of mutant forms of CYP19A1 were prepared by PCR mediated overlapping extension. Two rounds of PCR amplification were performed. The first round of PCR included two independent PCR using UF-CMV F and mutation specific R primers or UR-BGH R and mutation specific F primers to amplify coding region of CYP19A1 into two fragments. The PCR products were then treated with ExoSAP-IT from Affymetrix to eliminate the unincorporated primers and dNTPs. The mixture was incubated at 37° C. for 15 minutes followed by 80° C. for 15 minutes. Then, 10 ul of PCR products from each reaction were added to 30 ul of water. 5 ul of the diluted products were used as template to performed second round of PCR. In this PCR, UF and UR primers were added to the reaction to construct the linear expression cassette of CYP19A1 carrying the desired mutation. The amplified PCR product was run on agarose gels and purified using DNA gel purification kit from Qiagen.

### Construction of 3×ERE-Luc Reporter Construct

The oligonucleotides corresponding to the estrogen receptor binding site (estrogen responsive element) were designed and were shown as follow:

3XERE F:
(SEQ ID NO: 87)
TCGAGGTCAGGTCACAGTGACCTGATGTCAGGTCACAGTGACCTGAT
GTCAGGTCACAGTGACCTGATA
3XERE R:
(SEQ ID NO: 88)
AGCTTATCAGGTCACTGTGACCTGACATCAGGTCACTGTGACCTGAC
ATCAGGTCACTGTGACCTGACC

The oligonucleotides were re-suspended in TE buffer at 200 μM concentration. Equal amount of forward and reverse oligonucleotides were annealed into double strand form by incubated at 95° C. for 10 minutes. After the mixture cooled down to room temperature, 1 μl of double strand oligonucleotides was used as insert to ligate with lineralized pNL3.2 reporter construct (Promega). The lineralized vector was prepared by treating the DNA with XhoI and HindIII restriction enzymes. The ligated DNA was then transformed into Top10 competent cells (Invitrogen, Carlsbad, Calif.). The 3×ERE-luc reporter construct was confirmed by both restriction enzyme digestion and DNA sequencing.

### Transfection

For cell transfection experiments, HEK293 cells (ATCC) were plated at density of 4-8×104 cells per well (96-well plates) or 1-2×104 cells per well (384-well plates) in phenol red-free MEM containing 10% FBS and antibiotics. Either DNA plasmid or linear DNA was mixed with TransIT-293 transfection reagent (Minis Bio LLC). Once cells was trypsinized, DNA transfection mix was added. The cells were then incubated for 24 or 48 hours.

### Immunoblot Assay (Western Blotting)

Cells were collected 48 hours post-transfection, washed in PBS and lysed in ProteoJET mammalian cell lysis reagent (Fermentas) with protease and phosphatase inhibitors (Sigma). Lysates were centrifuged and supernatants were prepared for SDS-PAGE by addition of sample loading buffer (Bio-Rad). Lysates were subjected to 4-12% PAGE (Bio-Rad) and transferred to Immun-Blot PVDF membrane (Bio-Rad) per manufacturer's recommendations. Membranes were blocked in 5% milk/TPBT at room temperature for 1 hour. Membranes were probed with anti-ESR1 (Santa Cruz).

### ESR1

The protein sequence for ESR1 is below:

NCBI Reference Sequence: NM_000125.3
Protein
(SEQ ID NO: 90)
MTMTLHTKASGMALLHQIQGNELEPLNRPQLKIPLERPLGEVYL
DSSKPAVYNYPEGAAYEFNAAAAANAQVYGQTGLPYGPGSEAAAFGSNGL
GGFPPLNSVSPSPLMLLHPPPQLSPFLQPHGQQVPYYLENEPSGYTVREA
GPPAFYRPNSDNRRQGGRERLASTNDKGSMAMESAKETRYCAVCNDYASG
YHYGVWSCEGCKAFFKRSIQGHNDYMCPATNQCTIDKNRRKSCQACRLRK
CYEVGMMKGGIRKDRRGGRMLKHKRQRDDGEGRGEVGSAGDMRAANLWPS
PLMIKRSKKNSLALSLTADQMVSALLDAEPPILYSEYDPTRPFSEASMMG
LLTNLADRELVHMINWAKRVPGFVDLTLHDQVHLLECAWLEILMIGLVWR
SMEHPGKLLFAPNLLLDRNQGKCVEGMVEIFDMLLATSSRFRMMNLQGEE
FVCLKSIILLNSGVYTFLSSTLKSLEEKDHIHRVLDKITDTLIHLMAKAG
LTLQQQHQRLAQLLLILSHIRHMSNKGMEHLYSMKCKNVVPLYDLLLEML
DAHRLHAPTSRGGASVEETDQSHLATAGSTSSHSLQKYYITGEAEGFPAT
V

The DNA sequence for ESR1 is below;

DNA Coding sequence (start codon to stop codon)
(SEQ ID NO: 89)
atgacc
atgaccctcc acaccaaagc atctgggatg gccctactgc atcagatcca agggaacgag
ctggagcccc tgaaccgtcc gcagctcaag atccccctgg agcggcccct gggcgaggtg
tacctggaca gcagcaagcc cgccgtgtac aactaccccg agggcgccgc ctacgagttc
aacgccgcgg ccgccgccaa cgcgcaggtc tacggtcaga ccggcctccc ctacggcccc
gggtctgagg ctgcggcgtt cggctccaac ggcctggggg gtttcccccc actcaacagc
gtgtctccga gcccgctgat gctactgcac ccgccgccgc agctgtcgcc tttcctgcag
ccccacggcc agcaggtgcc ctactacctg gagaacgagc ccagcggcta cacggtgcgc
gaggccggcc cgccggcatt ctacaggcca aattcagata atcgacgcca gggtggcaga
gaaagattgg ccagtaccaa tgacaaggga agtatggcta tggaatctgc caaggagact
cgctactgtg cagtgtgcaa tgactatgct tcaggctacc attatggagt ctggtcctgt
gagggctgca aggccttctt caagagaagt attcaaggac ataacgacta tatgtgtcca
gccaccaacc agtgcaccat tgataaaaac aggaggaaga gctgccaggc ctgccggctc
cgcaaatgct acgaagtggg aatgatgaaa ggtgggatac gaaaagaccg aagaggaggg
agaatgttga aacacaagcg ccagagagat gatggggagg gcaggggtga agtggggtct
gctggagaca tgagagctgc caacctttgg ccaagcccgc tcatgatcaa acgctctaag
aagaacagcc tggccttgtc cctgacggcc gaccagatgg tcagtgcctt gttggatgct
gagcccccca tactctattc cgagtatgat cctaccagac ccttcagtga agcttcgatg
atgggcttac tgaccaacct ggcagacagg gagctggttc acatgatcaa ctgggcgaag
agggtgccag gctttgtgga tttgaccctc catgatcagg tccaccttct agaatgtgcc
tggctagaga tcctgatgat tggtctcgtc tggcgctcca tggagcaccc agggaagcta
ctgtttgctc ctaacttgct cttggacagg aaccagggaa aatgtgtaga gggcatggtg
gagatcttcg acatgctgct ggctacatca tctcggttcc gcatgatgaa tctgcaggga
gaggagtttg tgtgcctcaa atctattatt ttgcttaatt ctggagtgta cacatttctg

### CYP19A1

The protein sequence for CYP19A1 is below;

NCBI Reference Sequence: NM_000103.3
Protein
(SEQ ID NO: 92)
MVLEMLNPIHYNITSIVPEAMPAATMPVLLLTGLFLLVWNYEGT
SSIPGPGYCMGIGPLISHGRFLWMGIGSACNYYNRVYGEFMRVWISGEET
LIISKSSSMFHIMKHNHYSSRFGSKLGLQCIGMHEKGIIFNNNPELWKIT
RPFFMKALSGPGLVRMVIVCAESLKTHLDRLEEVINESGYVDVLILLRRV
MLDISNTLFLRIPLDESAIVVKIQGYFDAWQALLIKPDIFFKISWLYKKY
EKSVKDLKDAIEVLIAEKRRRISTEEKLEECMDFATELILAEKRGDLTRE
NVNQCILEMLIAAPDTMSVSLFFMLFLIAKHPNVEEAIIKEIQTVIGERD
IKIDDIQKLKVMENFIYESMRYQPVVDLVMRKALEDDVIDGYPVKKGTNI
ILNIGRMHRLEFFPKPNEFTLENFAKNVPYRYFQPFGFGPRGCAGKYIAM
VMMKAILVILLRRFHVKILQGQCVESIQKIHDLSLHPDETKNMLEMIFTP
RNSDRCLEH

The DNA sequence for CYP19A1 is below:

DNA Coding sequence (start codon to stop codon)
(SEQ ID NO: 91)
atggtttt ggaaatgctg aacccgatac attataacat
caccagcatc gtgcctgaag ccatgcctgc tgccaccatg ccagtcctgc tcctcactgg
cctttttctc ttggtgtgga attatgaggg cacatcctca ataccaggtc ctggctactg
catgggaatt ggacccctca tctcccacgg cagattcctg tggatgggga tcggcagtgc
ctgcaactac tacaaccggg tatatggaga attcatgcga gtctggatct ctggagagga
aacactcatt atcagcaagt cctcaagtat gttccacata atgaagcaca atcattacag
ctctcgattc ggcagcaaac ttgggctgca gtgcatcggt atgcatgaga aaggcatcat
atttaacaac aatccagagc tctggaaaac aactcgaccc ttctttatga aagctctgtc
aggccccggc cttgttcgta tggtcacagt ctgtgctgaa tccctcaaaa cacatctgga
caggttggag gaggtgacca atgaatcggg ctatgtggac gtgttgaccc ttctgcgtcg
tgtcatgctg gacacctcta acacgctctt cttgaggatc cctttggacg aaagtgctat
cgtggttaaa atccaaggtt attttgatgc atggcaagct ctcctcatca aaccagacat
cttctttaag atttcttggc tatacaaaaa gtatgagaag tctgtcaagg atttgaaaga
tgccatagaa gttctgatag cagaaaaaag acgcaggatt tccacagaag agaaactgga
gactttgcca ctgagttgat tttagcagag aaacgtggtg acctgacaag
agagaatgtg aaccagtgca tattggaaat gctgatcgca gctcctgaca ccatgtctgt
ctctttgttc ttcatgctat ttctcattgc aaagcaccct aatgttgaag aggcaataat
aaaggaaatc cagactgtta ttggtgagag agacataaag attgatgata tacaaaaatt
aaaagtgatg gaaaacttca tttatgagag catgcggtac cagcctgtcg tggacttggt
catgcgcaaa gccttagaag atgatgtaat cgatggctac ccagtgaaaa aggggacaaa
cattatcctg aatattggaa ggatgcacag actcgagttt ttccccaaac ccaatgaatt
tactcttgaa aattttgcaa agaatgttcc ttataggtac tttcagccat ttggctttgg
gccccgtggc tgtgcaggaa agtacatcgc catggtgatg atgaaagcca tcctcgttac
acttctgaga cgattccacg tgaagacatt gcaaggacag tgtgttgaga gcatacagaa
gatacacgac ttgtccttgc acccagatga gactaaaaac atgctggaaa tgatctttac
cccaagaaac tcagacaggt gtctggaaca ctag

What is claimed is:

1. A method of determining whether an estrogen receptor variant is sensitive to treatment with an estrogen receptor inhibitor in a cell, comprising the steps of:
a) preparing a cDNA encoding said estrogen receptor variant;
b) preparing a linear expression cassette containing the estrogen receptor variant cDNA;
c) transfecting said prepared linear expression cassette containing said estrogen receptor variant cDNA in an assay cell having a stably integrated signal expression construct comprising a reporter gene cDNA linked to an estrogen response element sequence;
d) exposing said transfected cell to estrogen, wherein said estrogen complexes with said estrogen receptor variant which thereby binds to said estrogen response element sequence on the signal construct and generates a signal;
e) exposing said transfected cell with an estrogen receptor inhibitor; and
f) determining whether said estrogen receptor variant is sensitive to treatment with said estrogen receptor inhibitor by measuring a change in signal.

2. The method of claim 1, wherein said estrogen receptor variant contains a missense mutation, insertion, or deletion.

3. The method of claim 1, wherein step c) and step d) are performed simultaneously.

4. The method of claim 1 wherein steps a) through f) are performed in 32 hours or less.

5. The method of claim 1, wherein said estrogen receptor inhibitor is Raloxifene, Tamoxifen, Toremifene, or Fulvestrant.

6. The method of claim 1, wherein said reporter gene cDNA encodes luciferase, said signal is light emission produced upon addition of a substrate for said luciferase, and a decrease in light emission relative to a control without estrogen inhibitor is indicative of the variant being sensitive to the inhibitor.

7. The method of claim 1 wherein said estrogen receptor variant is obtained from a patient's biological sample consisting of blood, serum, and tumor tissue.

8. The method of claim 1, wherein said estrogen receptor variant is transiently transfected.

9. A method of determining the activity of an estrogen receptor variant, comprising the steps of:
a) preparing a cDNA encoding said estrogen receptor variant;
b) preparing a linear expression cassette containing the estrogen receptor variant cDNA;
c) transfecting said prepared linear expression cassette containing said estrogen receptor variant cDNA in a cell having a stably integrated signal expression construct comprising a reporter gene cDNA linked to an estrogen response element sequence;
d) exposing said transfected cell to an estrogen, wherein said estrogen complexes with said estrogen receptor variant which thereby binds to said estrogen response element sequence on the signal construct and generates a signal;
e) determining the signal activity, wherein a change in signal activity relative to wild type estrogen receptor is indicative of a change in activity of said estrogen receptor variant.

10. The method of claim 9, wherein said estrogen receptor variant contains a missense mutation, insertion, or deletion.

11. The method of claim 10, wherein steps c) and d) are performed simultaneously.

12. The method of claim 10, wherein steps a) through f) are performed in 32 hours or less.

13. The method of claim 9, wherein said estrogen receptor inhibitor is Raloxifene, Tamoxifen, Toremifene, or Fulvestrant.

14. The method of claim 9, wherein said reporter gene cDNA encodes luciferase, said signal is light emission produced upon addition of a substrate for said luciferase, and said change in signal is an increase or decrease in light emission relative to wild type estrogen receptor, wherein an increase in light emission indicates an increase in estrogen receptor activity and a decrease in light signal indicates a decrease in estrogen receptor activity.

15. The method of claim 9, wherein said estrogen receptor variant is transiently transfected.

16. The method of claim 9 wherein said estrogen receptor variant is obtained from a patient's biological sample consisting of blood, serum, and tumor tissue.

17. A method of determining whether the activity of an aromatase variant is sensitive to treatment with an aromatase inhibitor in a cell, comprising the steps of:
a) preparing a cDNA encoding said aromatase variant;
b) preparing a linear expression cassette containing the aromatase variant cDNA;
c) transfecting said prepared linear expression cassette containing said aromatase variant cDNA in an assay cell having a stably integrated signal expression construct comprising a reporter gene cDNA linked to an estrogen response element sequence and said assay cell further having a stably integrated expression construct of estrogen receptor;
d) exposing said transfected cell to an androgen which is converted to estrogen by said aromatase variant, wherein said estrogen complexes with stably expressed estrogen receptor which thereby binds to said estrogen response element sequence on the signal construct and generates a signal;
e) exposing said transfected cell with an aromatase inhibitor; and
f) determining whether said aromatase variant is sensitive to treatment with said aromatase inhibitor by measuring a change in signal.

18. The method of claim 17, wherein said aromatase variant contains a missense mutation, insertion, or deletion.

19. The method of claim 18, wherein steps a) through f) are performed in 32 hours or less

20. The method of claim 17 wherein said androgen is testosterone.

21. The method of claim 17, wherein said aromatase inhibitor is anastrozole, exemestane, or letrozole,

22. The method of claim 17, wherein said reporter gene cDNA encodes luciferase, said signal is light emission produced upon addition of a substrate for said luciferase, and a decrease in light emission relative to a control without aromatase inhibitor is indicative of the variant being sensitive to the inhibitor.

23. The method of claim 17, wherein said aromatase variant is transiently transfected and steps c) and d) are performed simultaneously.

24. The method of claim 17 wherein said estrogen receptor variant is obtained from a patient's biological sample consisting of blood, serum, and tumor tissue.

25. A method of determining the activity of an aromatase variant, comprising the steps of:
a) preparing a cDNA encoding said aromatase variant;
b) preparing a linear expression cassette containing the aromatase variant cDNA;
c) transfecting said prepared linear expression cassette containing said aromatase variant cDNA in an assay cell having a stably integrated signal expression construct comprising a reporter gene cDNA linked to an estrogen response element sequence and said assay cell further having a stably integrated expression construct of estrogen receptor;
d) exposing said transfected cell to an androgen which is converted to estrogen by said aromatase variant, wherein said estrogen complexes with stably expressed estrogen receptor which thereby binds to said estrogen response element sequence on the signal construct and generates a signal;
e) determining the signal activity, wherein a change in signal activity relative to wild type aromatase is indicative of an increase or decrease in activity of said aromatase variant.

26. The method of claim 25, wherein said aromatase variant contains a missense mutation, insertion, or deletion.

27. The method of claim 26, wherein steps a) through f) are performed in 32 hours or less.

28. The method of claim 25 wherein said androgen is testosterone.

29. The method of claim 25, wherein said aromatase inhibitor is anastrozole, exemestane, or letrozole.

30. The method of claim 25, wherein said reporter gene cDNA encodes luciferase, said signal is light emission produced upon addition of a substrate for said luciferase, and said change in signal is an increase or decrease in light emission relative to wild type aromatase, wherein an increase in light emission indicates an increase in aromatase activity and a decrease in light signal indicates a decrease in aromatase activity.

31. The method of claim 25, wherein said aromatase variant is transiently transfected and steps c) and d) are performed simultaneously.

32. The method of claim 25 wherein said aromatase variant is obtained from a patient's biological sample consisting of blood, serum and tumor tissue.
